(19) United States
(12) Patent Application Publication
(10) Pub. No.: US 2026/0008797 A1
(43) Pub. Date: Jan. 8, 2026

(54) PROCESSES OF PREPARING PSILOCIN AND PSILOCYBIN

(71) Applicant: Terran Biosciences Inc., Miami Beach, FL (US)

(72) Inventors: Matthew Alexander James DUNCTON, Las Vegas, NV (US); Roy SWARINGEN, JR., Durham, NC (US)

(21) Appl. No.: 19/324,852

(22) Filed: Sep. 10, 2025

Related U.S. Application Data
(63) Continuation of application No. 19/026,788, filed on Jan. 17, 2025, now abandoned.
Application No. 19/026,788 is a continuation of application No. 18/691,804, filed on Mar. 13, 2024, now abandoned.
(60) Provisional application No. 63/244,150, filed on Sep. 14, 2021.
(60) Provisional application No. 63/282,655, filed on Nov. 23, 2021.

(51) Int. Cl. C07F 9/572; C07D 209/16

(52) U.S. Cl. CPC C07F 9/5728; C07D 209/16

(57) ABSTRACT

Described herein are processes for preparing psilocin and psilocybin and chemical intermediates used in the synthetic processes.

### CROSS-REFERENCE TO RELATED APPLICATIONS

This application is a continuation of U.S. patent application Ser. No. 19/026,788, filed on Jan. 17, 2025, which is a continuation of U.S. patent application Ser. No. 18/691,804, filed on Mar. 13, 2024, which is a U.S. National Phase Application, filed under 35 U.S.C. § 371, of International Application No. PCT/US2022/043463, filed on Sep. 14, 2022, which claims the benefit of U.S. Provisional Patent Application No. 63/244,150, filed on Sep. 14, 2021, and U.S. Provisional Patent Application No. 63/282,655, filed on Nov. 23, 2021, the content of each of which is incorporated by reference herein in its entirety.

### BACKGROUND

Psilocybin and psilocin are the two main hallucinogenic compounds of the “magic mushrooms” and both act as agonists at 5-hydroxytryptamine (5-HT)2A subtype receptors. Recently, psilocybin and psilocin have received attention to their therapeutic relevance. See, e.g., Dinis-Oliveira, Drug Metab. Rev., 2017, 49 (1):84-91. Psilocybin is primarily a psilocin prodrug that is dephosphorylated by alkaline phosphatase to active metabolite psilocin. There is a need for methods for preparing and manufacturing psilocybin and psilocin for therapeutic uses.

### SUMMARY

In one aspect, provided herein are processes of preparing Compound 1:

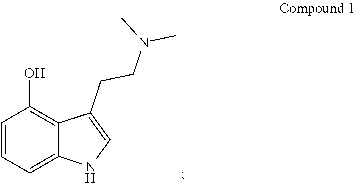

comprising (a-iii) contacting Compound D:

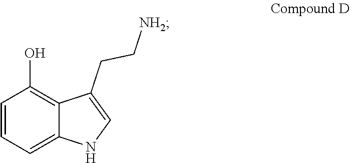 [SMILES: NCCc1c[nH]c2cccc(O)c12]

with a formaldehyde in the presence of a reducing agent, or with iodomethane in the presence of a base.

In certain embodiments, the reducing agent is a boron-containing reducing agent.

In certain embodiments, the reducing agent is NaCNBH₃ or NaBH₄.

In certain embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the base is sodium bicarbonate.

In certain embodiments, the processed further comprise (a-ii) contacting Compound C:

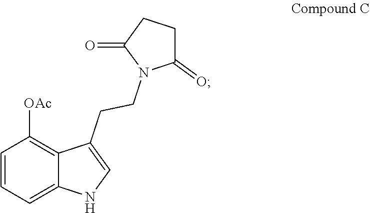 [SMILES: CC(=O)Oc1cccc2[nH]cc(CCN3C(=O)CCC3=O)c12]

with a base in the presence of a solvent to produce Compound D.

In certain embodiments, base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the base is potassium hydroxide.

In certain embodiments, the processes further comprise (a-i) contacting Compound B:

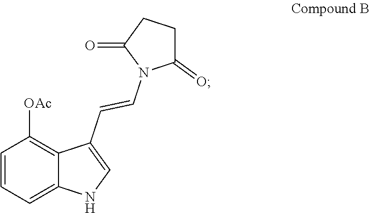 [SMILES: CC(=O)Oc1cccc2[nH]cc(/C=C/N3C(=O)CCC3=O)c12]

with a reducing agent in the presence of a catalyst and a solvent.

In certain embodiments, the reducing agent is hydrogen gas.

In certain embodiments, the catalyst is selected from Pd/C, Pd(OH)₂, Pd(OH)₂/C, Pd/Al₂O₃, Pd(OAc)₂/Et₃SiH, (PPh₃)₃RhCl, and PtO₂.

In certain embodiments, the processes further comprise (b) contacting Compound A:

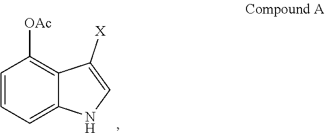

with N-vinyl succinimide in the presence of a catalyst, a base, and a solvent; wherein X is a halo.

In certain embodiments, the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]2Cl₂, Pd(amphos)Cl₂, Pd(dppf)Cl₂, Pd(dppf)Cl₂·CH₂Cl₂, Pd(dtbpf)Cl₂, Pd(MeCN)₄(BF4)₂, PdCl₂, XPhos-Pd-G3, Pd-PEPPSI™-IPr, Pd—PEPPSI™-SIPr, and Pd-PEPPSI™-IPent.

In certain embodiments, the catalyst is Pd(OAc)₂.

In certain embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the base is triethylamine.

In another aspect, also provided herein are processes of preparing Compound 1:

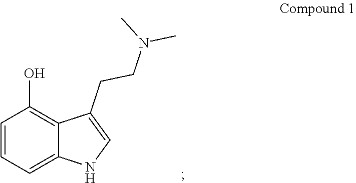

comprising (a) contacting Compound

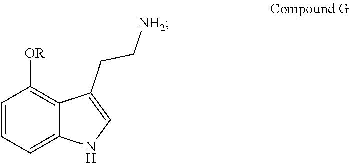

with a formaldehyde in the presence of a reducing agent, wherein R is hydrogen, acetyl, or benzyl.

In certain embodiments, the reducing agent is a boron-containing reducing agent.

In certain embodiments, the reducing agent is NaCNBH₃ or NaBH₄.

In certain embodiments, the reducing agent is formic acid.

In certain embodiments, when R is benzyl, further comprising (a′) contacting the product from step (a) with hydrogen gas in the presence of a catalyst.

In certain embodiments, the catalyst is selected from Pd/C, Pd(OH)₂, Pd(OH)₂/C, Pd/Al₂O₃, Pd(OAc)₂/Et₃SiH, (PPh₃)₃RhCl, and PtO₂.

In certain embodiments, the catalyst is selected from Pd/C,

In certain embodiments, the processes further comprise (b) contacting Compound F:

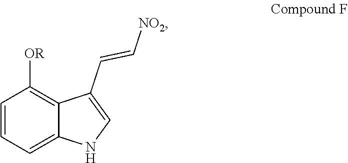

with a reducing agent in the presence of a solvent, wherein R is hydrogen, acetyl, or benzyl.

In certain embodiments, the reducing agent is selected from metal borohydride, lithium aluminum hydride, diisobutyl aluminum hydride, and sodium borohydride-iodine or boranes.

In certain embodiments, the reducing agent is lithium aluminum hydride.

In certain embodiments, the solvent is tetrahydrofuran, 2-Me-tetrahydrofuran, diethyl ether, dimethylformamide, dimethylsulfoxide, acetone, acetonitrile, 1,4-dioxane, or methyl tert-butyl ether, or combination thereof.

In certain embodiments, the solvent is tetrahydrofuran.

In certain embodiments, the processes further comprise: (c) contacting Compound E:

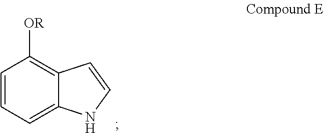

with (E)-N,N-dimethyl-2-nitroethen-1-amine in the presence of an acid to produce Compound F, wherein R is hydrogen, acetyl, or benzyl.

In certain embodiments, the acid is trifluoroacetic acid.

In certain embodiments, the processes further comprise: (c) contacting Compound E:

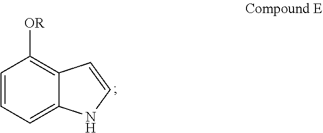

with POCl₃ in the presence of DMF to produce Compound F.

In certain embodiments, the processes further comprise: (c) contacting Compound E:

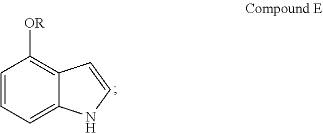

with Vilsmeir reagent (Me₂N⁺=CHCl Cl⁻) to produce Compound F.

In another aspect, also provided herein are processes of preparing Compound 1:

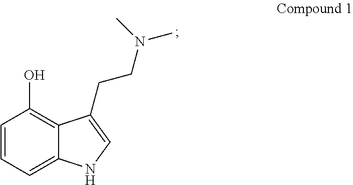

comprising (a) contacting Compound H:

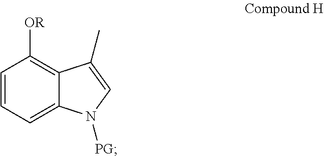

with Mannich salt Me₂N⁺=CH₂ X⁻ in the presence of a base, wherein X is I, Cl or CF₃CO₂, R is acetyl or methoxymethyl (MOM), and PG is a protecting group;
(b) removing the protecting group in Compound J:

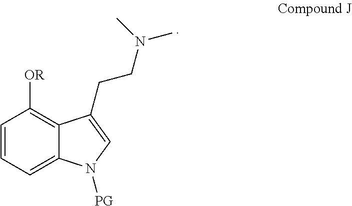

In certain embodiments, the protecting group is Boc, tosyl, or methoxymethyl (MOM).

In certain embodiments, the base is nBuLi, BuLi, MeMgBr, or R^{A}MgCl·LiCl, wherein R^{A} is alkyl or N-containing hetercyclyl.

In certain embodiments, the base is iPrMgCl·LiCl, 2,2,6,6-tetramethylpiperidine, or sBuMgCl·LiCl.

In certain embodiments, the base is a Grignard or a Turbo Grignard reagent.

In yet another aspect, also provided herein are processes of preparing Compound 1:

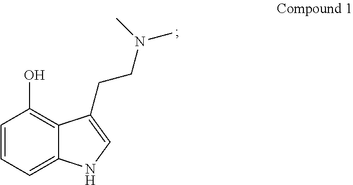

comprising (a) contacting Compound K:

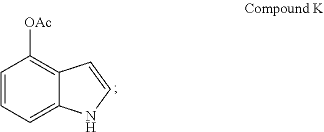 [SMILES: CC(=O)Oc1cccc2[nH]ccc12 ; ";"]

with a 1,1-dimethylaziridinium compound; and
(b) contacting Compound L:

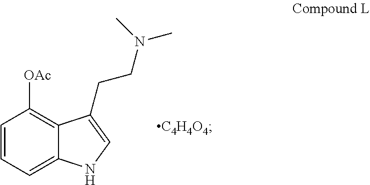 [SMILES: CC(=O)Oc1cccc2[nH]cc(CCN(C)C)c12]

with a base in the presence of a solvent.

In certain embodiments, the 1,1-dimethylaziridinium compound is 1,1-dimethylaziridinium BF₄⁻, ClO₄⁻, or OTf⁻.

In certain embodiments, the 1,1-dimethylaziridinium compound is 1,1-dimethylaziridinium BF4.

In certain embodiments, the 1,1-dimethylaziridinium compound is produced in situ by contacting Me₂NCH₂CH₂Cl·HCl and a second base under heating condition.

In certain embodiments, the second base is an inorganic base selected from Cs₂CO₃, K₂CO₃, Na₂CO₃, or NaHCO₃, or the second base is an organic base selected from piperidine, 1,8-diazabicyclo[5.4.0]undec-7-ene, N,N-diisopropylethylamine, and triethylamine.

In certain embodiments, the 1,1-dimethylaziridinium compound is produced in situ from Me₂NCH₂CH₂OMs under heating condition.

In certain embodiments, step (a) occurs at a temperature between 20° C. and 150° C.

In certain embodiments, step (a) occurs at a temperature between 50° C. and 120° C.

In certain embodiments, step (a) occurs at a temperature between 70° C. and 100° C.

In certain embodiments, the processes further comprise (b′) contacting with fumaric acid.

In certain embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the first base is potassium hydroxide.

In yet another aspect, also provided herein are processes of preparing Compound 1:

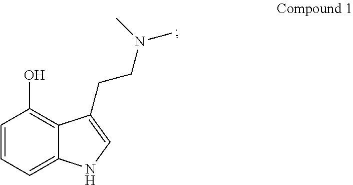

comprising (a) contacting Compound K:

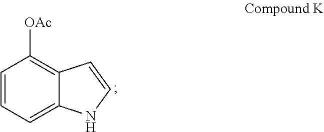 [SMILES: CC(=O)Oc1cccc2[nH]ccc12 ; ";"]

with aziridinium tetrafluoroborate and

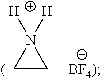

(b) contacting Compound M:

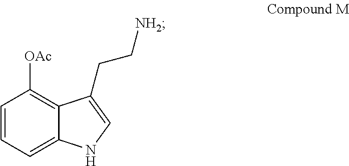 [SMILES: CC(=O)Oc1cccc2[nH]cc(CCN)c12]

with a formaldehyde in the presence of a reducing agent, or with iodomethane in the presence of a first base; and
(c) contacting Compound L:

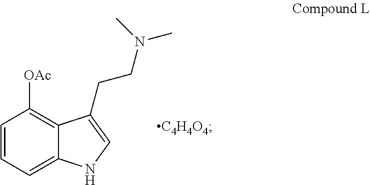 [SMILES: CC(=O)Oc1cccc2[nH]cc(CCN(C)C)c12]

with a second base in the presence of a solvent.

In certain embodiments, the processes further comprise (b′) contacting with fumaric acid.

In certain embodiments, in step (b) the reducing agent is a boron-containing reducing agent.

In certain embodiments, the reducing agent is NaCNBH₃ or NaBH₄.

In certain embodiments, the first base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the first base is sodium bicarbonate.

In certain embodiments, the second base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the second base is potassium hydroxide.

In yet another aspect, also provided herein are processes of preparing Compound 1:

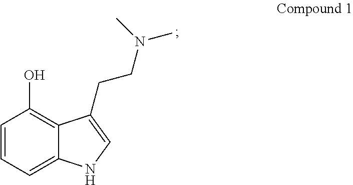

comprising (a) contacting Compound K:

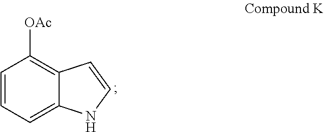 [SMILES: CC(=O)Oc1cccc2[nH]ccc12 ; ";"]

with a cyclic sulfamidate in the presence of a metal base; and
(b) contacting Compound M:

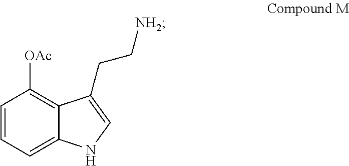 [SMILES: CC(=O)Oc1cccc2[nH]cc(CCN)c12]

with a formaldehyde in the presence of a reducing agent, or with iodomethane in the presence of a first base; and
(c) contacting Compound L:

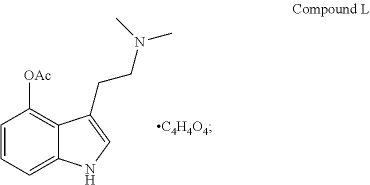 [SMILES: CC(=O)Oc1cccc2[nH]cc(CCN(C)C)c12]

with a second base in the presence of a solvent.

In certain embodiments, step (a) occurs at a temperature between 20° C. to 150° C.

In certain embodiments, step (a) occurs at a temperature between 50° C. to 120° C.

In certain embodiments, step (a) occurs at a temperature between 70° C. to 100° C.

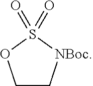 [SMILES: CC(C)(C)OC(=O)N1CCOS1(=O)=O]

In certain embodiments, the cyclic sulfamidate is

In certain embodiments, the metal base is MeMgBr.

In certain embodiments, step (a) occurs in the presence of MeMgBr and CuCl in DCM at −20° C. to 0° C.

In certain embodiments, the processes further comprise (a′) deprotecting Boc protecting group.

In certain embodiments, in step (b) the reducing agent is a boron-containing reducing agent.

In certain embodiments, the reducing agent is NaCNBH₃ or NaBH₄.

In certain embodiments, the first base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the first base is sodium bicarbonate.

In certain embodiments, the processes further comprise (b′) contacting with fumaric acid.

In certain embodiments, the second base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the second base is potassium hydroxide.

In yet another aspect, also provided herein are processes of preparing Compound 2:

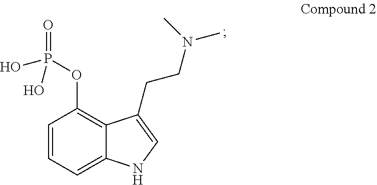 [SMILES: CN(C)CCc1c[nH]c2cccc(OP(=O)(O)O)c12 ; ";"]

comprising (a) contacting Compound K:

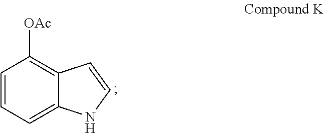 [SMILES: CC(=O)Oc1cccc2[nH]ccc12 ; ";"]

with 2-(dimethylamino)acetaldehyde or its derivative in the presence of a catalyst; and
(b) contacting Compound M:

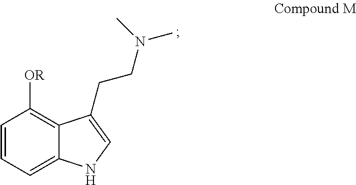

with POCl₃ in a solvent; wherein R is hydrogen or acetyl.

In certain embodiments, the processes further comprise: (c) contacting with water in the presence of a base in a solvent.

In certain embodiments, the base is triethylamine.

In certain embodiments, the catalyst is a Lewis acid catalyst.

The process of claim 76, wherein the Lewis acid catalyst is AlCl₃, BF₃OEt₂, NaBH₄, ZnCl₂, Zn(OTf)₂, Sc(OTf)₃, or CuCl₂.

In certain embodiments, the catalyst is a base.

In certain embodiments, the catalyst is MeMgBr combined with CuCl.

In certain embodiments, the 2-(dimethylamino)acetaldehyde derivative is (dimethylamino)acetaldehyde dimethyl acetal.

In certain embodiments, step (a) and step (b) occur in one-pot synthesis.

In yet another aspect, further provided herein are processes of preparing Compound 1:

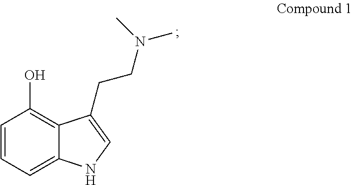

comprising (a) contacting Compound E:

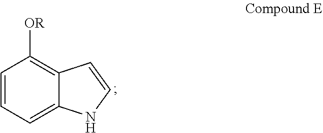

with 2-(dimethylamino) ethanol in the presence of a first catalyst and a first base, wherein R is hydrogen, acetyl, or benzyl; and
(b) contacting Compound N:

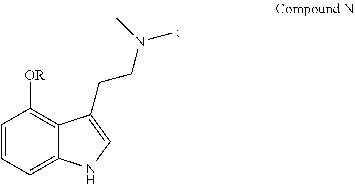

with hydrogen gas in the presence of a second catalyst, or with a second base.

In certain embodiments, the first catalyst is [Cp*IrCl₂]₂, Fe(II)Pc, or Cu(OAc)₂/dppm.

In certain embodiments, the first base is Cs₂CO₃, NaO^{t}Bu, KO^{t}Bu, K₂CO₃, Na₂CO₃, or NaHCO₃.

In certain embodiments, step (a) occurs at a temperature of between 50° C. and 170° C.

In certain embodiments, step (a) occurs at a temperature of between 85° C. and 150° C. In certain embodiments, the first base is sodium bicarbonate.

In certain embodiments, the second base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In certain embodiments, the second base is potassium hydroxide.

In certain embodiments, the second catalyst is selected from Pd/C, Pd(OH)₂, Pd(OH)₂/C, Pd/Al₂O₃, Pd(OAc)₂/Et₃SiH, (PPh₃)₃RhCl, and PtO₂.

In certain embodiments, the second catalyst is Pd/C.

In yet another aspect, further provided herein are processes of preparing Compound 1:

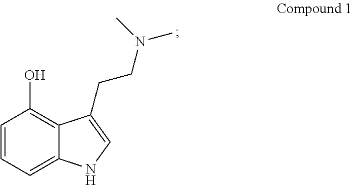

comprising (a) contacting Compound E:

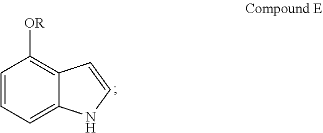

with chloroacetaldehyde and Me₂NH in the presence of an acid, wherein R is hydrogen, acetyl, or benzyl; and
(b) contacting Compound O:

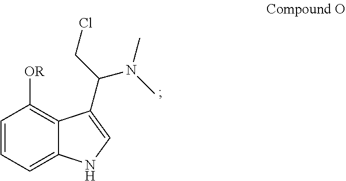

with a reducing agent.

In certain embodiments, the acid is an organic acid.

In certain embodiments, the acid is acetic acid or propionic acid.

In certain embodiments, step (a) occurs at a temperature of about 0° C.

In certain embodiments, the reducing agent is a boron-containing reducing agent.

In certain embodiments, the reducing agent is NaCNBH₃ or NaBH₄.

In yet another aspect, further provided herein are processes of preparing Compound 2:

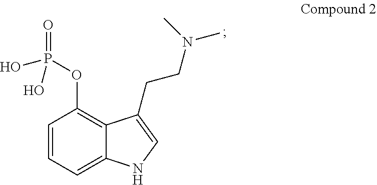 [SMILES: CN(C)CCc1c[nH]c2cccc(OP(=O)(O)O)c12 ; ";"]

comprising:
(a) preparing Compound 1 according to any one of the processes described herein; and
(b) contacting Compound 1 with POCl₃ and a base in a solvent.

In certain embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the base is triethylamine.

In certain embodiments, the solvent is tetrahydrofuran, 2-Me-tetrahydrofuran, diethyl ether, dimethylformamide, dimethylsulfoxide, acetone, acetonitrile, 1,4-dioxane, or methyl tert-butyl ether, or combination thereof.

In certain embodiments, the solvent is tetrahydrofuran.

In certain embodiments, step (b) occurs at a temperature of about 0° C.

In yet another aspect, further provided herein are processes of preparing Compound 2:

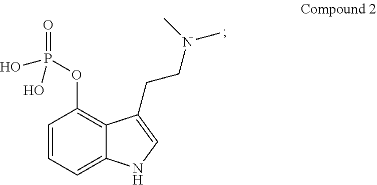 [SMILES: CN(C)CCc1c[nH]c2cccc(OP(=O)(O)O)c12 ; ";"]

comprising:
(a) preparing Compound 1 according to any one of the processes described herein; and
(b) contacting Compound 1 with the Ψ-reagent, (2R,3aS,6R,7aS)-2-((4-bromophenyl)thio)-6-isopropyl-3a-methylhexahydrobenzo[d][1,3,2]oxathiaphosphole 2-oxide in the presence of base in a solvent.

In certain embodiments, a stoichiometric amount of the Ψ-reagent is contacted.

In certain embodiments, between 1.0 and 10.0 equivalents of the Ψ-reagent is contacted.

In certain embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

In certain embodiments, the base is 1,8-diazabicyclo[5.4.0]undec-7-ene.

In certain embodiments, between 1.0 and 10.0 equivalents of 1,8-diazabicyclo[5.4.0]undec-7-ene is present.

In certain embodiments, the solvent is tetrahydrofuran, 2-Me-tetrahydrofuran, dimethylformamide, dimethylsulfoxide, acetonitrile, dichloromethane, dichloroethane, chloroform, or toluene.

In certain embodiments, the solvent is dimethylformamide.

In certain embodiments, step (b) occurs at a temperature of between room temperature and 150° C.

In certain embodiments, further comprising (c) contacting water in the presence of acetonitrile.

In yet another aspect, further provided herein are processes of preparing Compound 2:

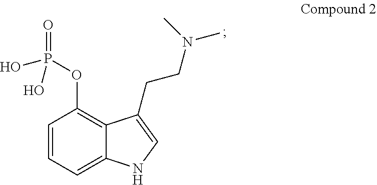 [SMILES: CN(C)CCc1c[nH]c2cccc(OP(=O)(O)O)c12 ; ";"]

comprising:
(a) preparing Compound 1 according to any one of the processes described herein; and
(b) contacting Compound 1 with phosphoric acid, Bu₃N, in the presence of a base in a solvent.

In certain embodiments, between 1.0 and 10.0 equivalents of phosphoric acid is contacted.

In certain embodiments, the base is a nucleophilic base.

In certain embodiments, the nucleophilic base is N-butylimidazole or 4-(N,N-dimethylamino)pyridine.

In certain embodiments, a catalytic amount of the nucleophilic base is present.

In certain embodiments, up to a stoichiometric amount of the nucleophilic base is present.

In certain embodiments, between 10 mol % to 100 mol % of the nucleophilic base is present.

In certain embodiments, a stoichiometric amount of Bu₃N is present.

In certain embodiments, between 1.0 and 10.0 equivalents of Bu₃N is present.

In certain embodiments, the solvent is DMF, 1:1 (v/v) mixture of DMF and nitroethane, or o-xylene.

In certain embodiments, wherein step (b) occurs at a temperature of between room temperature and 150° C.

In certain embodiments, (b) occurs at a preferred temperature of azeotropic reflux with removal of water.

In certain embodiments, the removal of water uses 3 Å molecular sieves.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

As used in the specification and appended claims, unless specified to the contrary, the following terms have the meaning indicated below.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range.

The term “comprising” (and related terms such as “comprise” or “comprises” or “having” or “including”) is not intended to exclude that which in other certain embodiments, for example, an embodiment of any composition of matter, composition, method, or process, or the like, described herein, “consist of” or “consist essentially of” the described features.

### Processes for Preparation of Psilocybin and Psilocin

Disclosed herein are methods of manufacturing psilocin (4-hydroxy-N,N-dimethyltryptamine), psilocybin, and analogs thereof. Synthetic methods provided herein for preparation of psilocin and psilocybin, or a protected form thereof are summarized in Scheme 1 below. Some of the approaches may require protection at the indole nitrogen atom (i.e. NR rather than NH) and/or at the oxygen atom. See, e.g., J. Fricke et al., Chem. Eur. J., 2019, 25, 897.

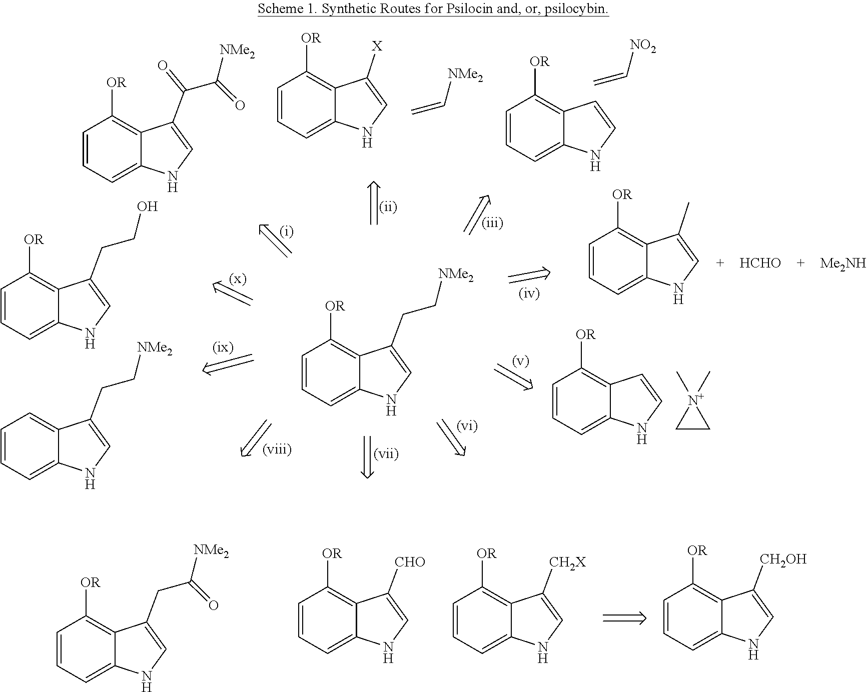

### 1. Friedel-Crafts Acylation/Amide Formation/Reduction

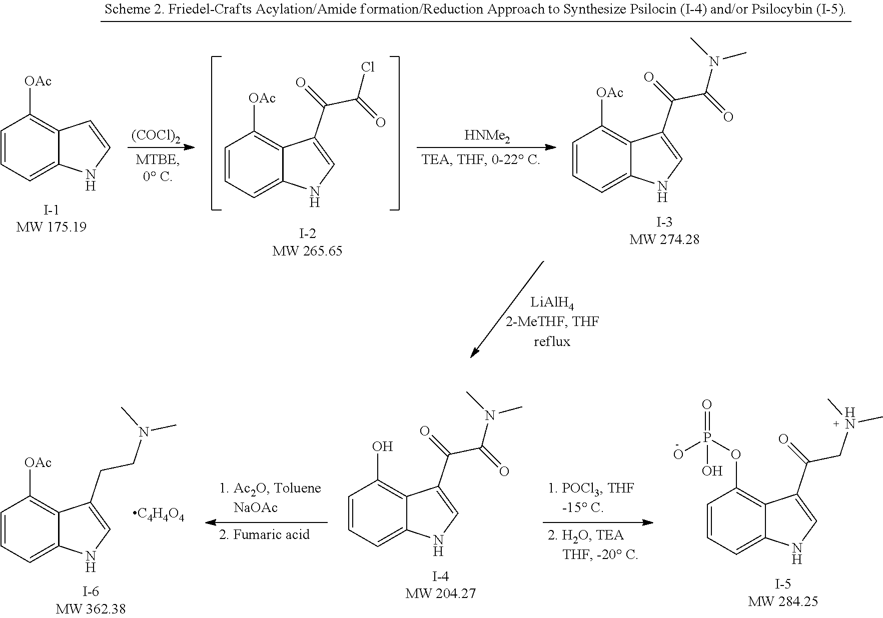

Prior syntheses of psilocin and improved procedures using a Friedel-Crafts acylation/amide formation/reduction synthetic route have been reported by Kargbo et al. (see, e.g., Scheme 2). See, e.g., Kargbo et al., ACS Omega, 2020, 5, 16959-16966 and references cited therein. For example, psilocin (I-4) can be synthesized from the starting material I-1 via intermediate I-2.

Psilocin has been successfully synthesized using this reported synthetic route. In some embodiments, addition of 10 volumes of heptane, rather than 5 volumes, results in a better precipitation of product and easier isolation. In some embodiments, pre-mixed Et₃N (triethylamine, TEA) and Me₂NH are used for the reaction of preparing 3-(2-(dimethylamino)-2-oxoacetyl)-1H-indol-4-yl acetate (I-3).

### Exemplary Reaction to Produce 3-(2-chloro-2-oxoacetyl)-1H-indol-4-yl acetate (I-2)

A 3-neck 500 mL round bottom flask equipped with a mechanical stirrer and thermometer was purged with nitrogen. Oxalyl chloride (17.39 g, 11.99 mL, 1.2 eq., 137.0 mmol) was charged, followed by methyl tert-butyl ether (MTBE) (100 mL) and the resulting solution was cooled to 0-5° C. A solution of 1H-indol-4-yl acetate (I-1, 20.00 g, 1 eq., 114.2 mmol) dissolved in 120 mL MTBE was charged to the cold oxalyl chloride solution, keeping the internal temperature 5-10° C. The addition was complete in 35 min. The formation of a yellow precipitate was observed 30 minutes after complete addition and increased over time. After stirring at 0-5° C. for 2 h, an analytical sample from the reaction mixture was diluted with methanol (to form the methyl ester). LC-MS analysis indicated completion of the reaction. The reaction was stirred for an additional hour before heptane (200 mL) was charged dropwise to the cold suspension, keeping the temperature at 5-10° C. The addition was complete within 10 min to give a dense yellow suspension. This suspension was aged with stirring at 0-5° C. for 1 hour and 15 min, then filtered, and the filter cake was washed with 125 mL of room temperature 1:4 MTBE/Heptane mixture. The filter cake was dried under vacuum for 30 min to give 3-(2-chloro-2-oxoacetyl)-1H-indol-4-yl acetate (25.80 g, 86.5%) as a yellow solid.

### Exemplary Reaction to Produce 3-(2-(dimethylamino)-2-oxoacetyl)-1H-indol-4-yl acetate (I-3)

In a 500 mL 3-neck round-bottom flask, under a nitrogen atmosphere, 3-(2-chloro-2-oxoacetyl)-1H-indol-4-yl acetate (I-2, 12.08 g, 1 eq., 45.47 mmol) was dissolved in THF (130 mL) and the solution was cooled to 5-10° C. A solution consisting of pre-mixed dimethylamine (2.0 M in THF) (2.666 g, 29.56 mL, 2 molar, 1.3 Eq, 59.12 mmol) 10 mL THF and Et₃N (5.982 g, 8.24 mL, 1.3 eq., 59.12 mmol) was charged over 40 min via dropping funnel, keeping the internal temperature below 10° C. A cream suspension formed. Note that amine hydrochloride formed in the headspace above the reaction suspension during the addition, and mostly redissolved over time. The headspace was washed with 10 mL THF. The ice bath was removed, and the resulting cream suspension was stirred at room temperature overnight. Heptane (300 mL) was added over 5 min and the suspension was cooled in an ice bath to 5° C. and stirred for 2 h before filtration. The filter cake was washed with Heptane (25 mL×2) and the filter cake was dried under vacuum suction in the filter funnel for 20 min under a nitrogen blanket to avoid dis-coloration to give 22.89 g of a tan solid.

### Recrystallization:

Under a nitrogen atmosphere, the 22.80 g tan solid was transferred to a 3-neck 500 mL round bottom flask equipped with a mechanical stirrer and reflux condenser. Isopropyl alcohol (IPA) (120 mL) was charged and the suspension was heated to achieve an oil bath temperature of 90° C. After 45 min of stirring at this temperature, complete dissolution was achieved. After 20 minutes of stirring with complete dissolution, the heat was turned off and the mixture was allowed to cool to room temperature overnight. The suspension was cooled to 0-5° C., and the suspension was stirred for 1 h, then filtered (Buchner funnel), and the filter cake washed with 0° C. cooled IPA (20 mL). The filter cake was dried under suction vacuum on the filter funnel for 15 min until no more droplets of solvent were observed. The filter cake was washed successively with 0° C. cooled water (20 mL×3), then washed with 0° C. cooled heptane (20 mL) and dried under suction vacuum for 45 min. The 9.54 g tan solid was placed under vacuum pump for two days to give 3-(2-(dimethylamino)-2-oxoacetyl)-1H-indol-4-yl acetate (I-3, 8.92 g, 71.5%) as a tan solid.

### Exemplary Reaction to Produce Psilocin (I-4)

Lithium aluminium hydride in THF (2.4M; 78 ml, 187 mmol) was added over 1 h to a pre-heated (62° C.) stirred suspension of 3-(2-(dimethylamino)-2-oxoacetyl)-1H-indol-4-yl acetate (I-3, 18.0 g, 66 mmol) in 2-methyltetrahydrofuran (200 mL) under nitrogen, maintaining a batch temperature at 60-70° C. The yellow suspension was stirred at 75-76° C. (gentle reflux) for 4 h, then cooled to 28° C. H₂O (15.0 ml) was added over 30 min with stirring and the mixture was stored overnight. The mixture was charged with THF-MeOH (135 mL: 15 mL), then stirred for 5 min, and Na₂SO₄ (32.32 g) and silica gel (8.64 g) were added. The mixture was stirred for 15 min, then filtered under a nitrogen envelope. The residue was washed through three times with THF-methanol (180 mL: 20 mL). The combined filtrates were evaporated at 38° C. to about 50 mL total volume, then diluted with heptane (180 mL). The mixture was re-evaporated to about 50 mL total volume and MTBE (50 mL) was added. The mixture was stirred for 15 min at 40° C., cooled to ambient temperature and filtered in a nitrogen envelope. The cake was deliquored and pulled dry under nitrogen for 2 h, then discharged as a faintly olive-green powder to give psilocin (10.86 g). A second crop (0.38 g) was recovered from the mother liquor. The overall yield was 83.4%.

However, new synthetic procedures for preparing psilocybin (1-5) from psilocin (I-4) are provided herein.

### Exemplary Reactions to Produce Psilocybin (1-5)

In certain embodiments, as shown in Scheme 2A below, the Ψ-reagent, (2R,3aS,6R,7aS)-2-((4-bromophenyl)thio)-6-isopropyl-3a-methylhexahydrobenzo[d][1,3,2]oxathiaphosphole 2-oxide is used for the phosphorylation of psilocin (I-4) to synthesize psilocybin (I-5). Reference is made to Ociepa et al., Org. Lett., 2021, 23, 9337-9342 and references cited therein. In certain embodiments, the Ψ-reagent is be used in a stoichiometric amount, or an excess amount (up to 10 equivalents). In certain embodiments, the preferred quantity of Ψ-reagent is between 1.0 and 10.0 equivalents. In certain embodiments, the preferred quantity of DBU is between 1.0 and 10.0 equivalents. In certain embodiments, the preferred temperature is room temperature to 150° C. In certain embodiments, the preferred solvent for the reaction ais selected from DMF, MeCN, DCM, 1,2-DCE, CHCl3 and toluene. In certain embodiments, if a non-water miscible solvent is used, then MeCN should also be added for the hydrolysis step.

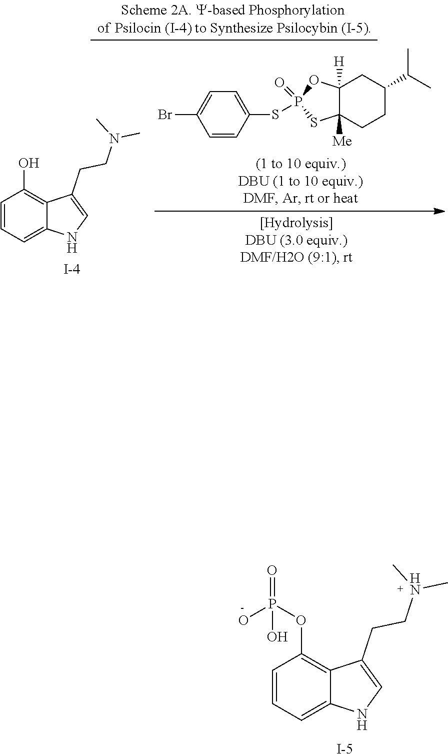

In one aspect, provided herein are processes of preparing Compound 2 (psilocybin),

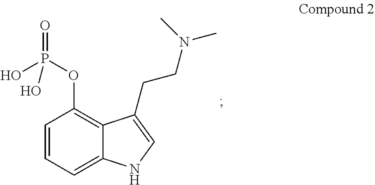 [SMILES: CN(C)CCc1c[nH]c2cccc(OP(=O)(O)O)c12]

comprising:
(a) preparing Compound 1 according to the processes described herein;
(b) contacting Compound 1 with the Ψ-reagent, (2R,3aS,6R,7aS)-2-((4-bromophenyl)thio)-6-isopropyl-3a-methylhexahydrobenzo[d][1,3,2]oxathiaphosphole 2-oxide in the presence of base in a solvent.

In some embodiments, a stoichiometric amount of the Ψ-reagent is contacted. In some embodiments, between 1.0 and 10.0 equivalents of the Ψ-reagent is contacted.

In some embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium hydroxide. In some embodiments, the base is cesium carbonate. In some embodiments, the base is potassium carbonate. In some embodiments, the base is sodium bicarbonate. In some embodiments, the base is sodium bicarbonate. In some embodiments, the base is piperidine. In some embodiments, the base is, 8-diazabicyclo[5.4.0]undec-7-ene. In some embodiments, the base is N,N-diisopropylethylamine. In some embodiments, the base is triethylamine.

In some embodiments, the base is 1,8-diazabicyclo[5.4.0]undec-7-ene. In some embodiments, between 1.0 and 10.0 equivalents of 1,8-diazabicyclo[5.4.0]undec-7-ene is present.

In some embodiments, the solvent is tetrahydrofuran, 2-Me-tetrahydrofuran, dimethylformamide, dimethylsulfoxide, acetonitrile, dichloromethane, dichloroethane, chloroform, or toluene. In some embodiments, the solvent is tetrahydrofuran. In some embodiments, the solvent is 2-Me-tetrahydrofuran. In some embodiments, the solvent is dimethylformamide. In some embodiments, the solvent is dimethylsulfoxide. In some embodiments, the solvent is acetonitrile. In some embodiments, the solvent is dichloromethane. In some embodiments, the solvent is dichloroethane. In some embodiments, the solvent is chloroform. In some embodiments, the solvent is toluene.

In some embodiments, step (b) occurs at a temperature of between room temperature and 150° C.

In some embodiments, the processes further comprise (c) contacting water in the presence of acetonitrile.

In certain embodiments, in an alternative process as shown in Scheme 2B below, psilocybin (1-5) is synthesized by dehydrative condensation of phosphoric acid. Reference is made to Sakakura et al., Org. Lett. 2005, 7, 1999-2002. The product is synthesized from a mixture of phosphoric acid (1 to 10 equivalent) and 3-(2-(dimethylamino)ethyl)-1H-indol-4-ol (I-4, 1 equivalent) in the presence of Bu₃N and nucleophilic bases such as N-butylimidazole or 4-(N,N-dimethylamino)pyridine. In certain embodiments, Bu₃N is used in a stoichiometric amount, or an excess amount (up to 10 equivalents). In certain embodiments, N-butylimidazole is used in catalytic quantities, up to stoichiometric quantities, with a preferred amount of 10 mol % to 100 mol %. In certain embodiments, the preferred solvent is DMF, 1:1 (v/v) mixture of DMF and nitroethane, o-xylene, or other solvents. In certain embodiments, the temperature is room temperature to 150° C., with a preferred temperature of azeotropic reflux with the removal of water using 3A molecular sieves.

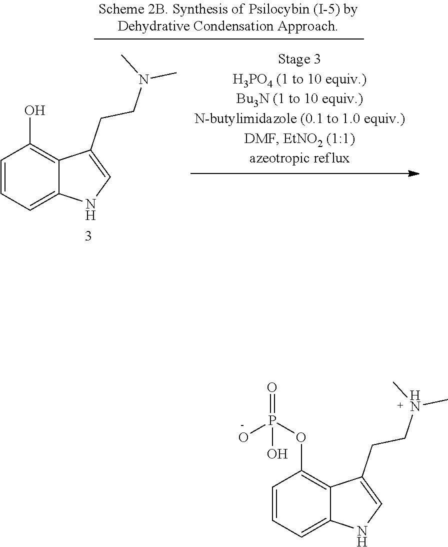

In another aspect, provided herein are processes of preparing Compound 2 (psilocybin),

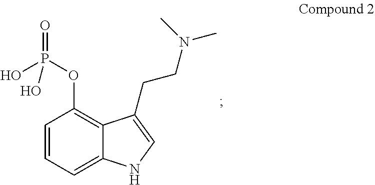 [SMILES: CN(C)CCc1c[nH]c2cccc(OP(=O)(O)O)c12]

comprising:
(a) preparing Compound 1 according to the processes described herein;
(b) contacting Compound 1 with phosphoric acid, Bu₃N, in the presence of a base in a solvent.

In some embodiments, between 1.0 and 10.0 equivalents of phosphoric acid is contacted.

In some embodiments, the base is a nucleophilic base. In some embodiments, the nucleophilic base is N-butylimidazole or 4-(N,N-dimethylamino)pyridine.

In some embodiments, a catalytic amount of the nucleophilic base is present. In some embodiments, up to a stoichiometric amount of the nucleophilic base is present. In some embodiments, between 10 mol % to 100 mol % of the nucleophilic base is present.

In some embodiments, a stoichiometric amount of Bu₃N is present. In some embodiments, wherein between 1.0 and 10.0 equivalents of Bu₃N is present.

In some embodiments, the solvent is DMF, 1:1 (v/v) mixture of DMF and nitroethane, or o-xylene. In some embodiments, the solvent is DMF. In some embodiments, the solvent is 1:1 (v/v) mixture of DMF and nitroethane. In some embodiments, the solvent is o-xylene.

In some embodiments, step (b) occurs at a temperature of between room temperature and 150° C. In some embodiments, step (b) occurs at a preferred temperature of azeotropic reflux with removal of water. In some embodiments, the removal of water uses 3 Å molecular sieves.

### 2. Heck-Type Reaction of Vinyldimethylamine with an Indole

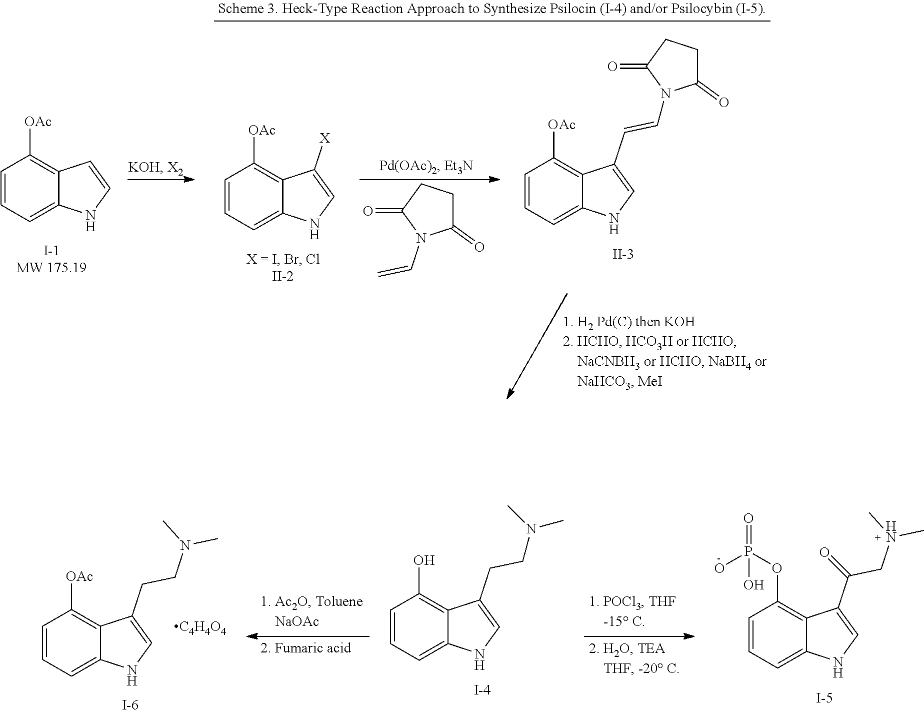

In some embodiments, psilocin and psilocybin can be synthesized using the synthetic route illustrated in Scheme 3 using a Heck-type reaction.

In one aspect, provided herein are processes for preparing Compound 1 (psilocin):

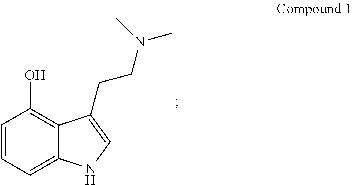

comprising (a-iii) contacting Compound D:

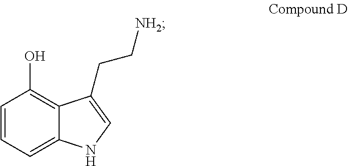 [SMILES: NCCc1c[nH]c2cccc(O)c12]

with a formaldehyde in the presence of a reducing agent, or with iodomethane in the presence of a base.

In some embodiments, the reducing agent is a boron-containing reducing agent. In some embodiments, the reducing agent is NaCNBH₃ or NaBH₄.

In some embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium hydroxide. In some embodiments, the base is cesium carbonate. In some embodiments, the base is potassium carbonate. In some embodiments, the base is sodium bicarbonate. In some embodiments, the base is sodium bicarbonate. In some embodiments, the base is piperidine. In some embodiments, the base is, 8-diazabicyclo[5.4.0]undec-7-ene. In some embodiments, the base is N,N-diisopropylethylamine. In some embodiments, the base is triethylamine.

In some embodiments, the processes further comprise: (a-ii) contacting Compound C:

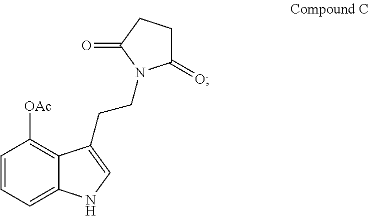 [SMILES: CC(=O)Oc1cccc2[nH]cc(CCN3C(=O)CCC3=O)c12]

with a base in the presence of a solvent to produce Compound D.

In some embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium hydroxide. In some embodiments, the base is cesium carbonate. In some embodiments, the base is potassium carbonate. In some embodiments, the base is sodium bicarbonate. In some embodiments, the base is sodium bicarbonate. In some embodiments, the base is piperidine. In some embodiments, the base is, 8-diazabicyclo[5.4.0]undec-7-ene. In some embodiments, the base is N,N-diisopropylethylamine. In some embodiments, the base is triethylamine.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the processes further comprise: (a-i) contacting Compound B:

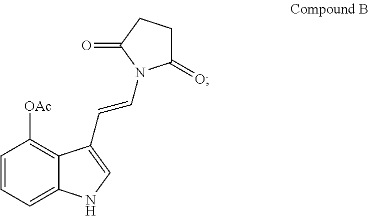 [SMILES: CC(=O)Oc1cccc2[nH]cc(/C=C/N3C(=O)CCC3=O)c12]

with a reducing agent in the presence of a catalyst and a solvent.

In some embodiments, the reducing agent is hydrogen gas.

In some embodiments, the catalyst is selected from Pd/C, Pd(OH)₂, Pd(OH)₂/C, Pd/Al₂O₃, Pd(OAc)₂/Et₃SiH, (PPh₃)₃RhCl, and PtO₂. In some embodiments, the catalyst is Pd/C. In some embodiments, the catalyst is Pd(OH)₂. In some embodiments, the catalyst is Pd(OH)₂/C. In some embodiments, the catalyst is Pd/Al₂O₃. In some embodiments, the catalyst is Pd(OAc)₂/Et₃SiH. In some embodiments, the catalyst is (PPh₃)₃RhCl. In some embodiments, the catalyst is PtO₂.

In some embodiments, the processes further comprise: (b) contacting Compound A:

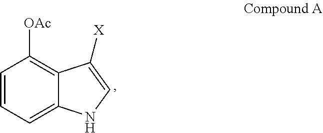

with N-vinyl succinimide in the presence of a catalyst, a base, and a solvent; wherein X is a halo.

In some embodiments, the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(MeCN)₄ (BF₄)₂, PdCl₂, XPhos-Pd-G3, Pd-PEPPSI™-IPr, Pd—PEPPSI™-SIPr, and Pd-PEPPSI™-IPent. In some embodiments, the catalyst is Pd(acac)₂. In some embodiments, the catalyst is [Pd(allyl)Cl]₂. In some embodiments, the catalyst is Pd(MeCN)₂Cl₂. In some embodiments, the catalyst is Pd(dba)₂. In some embodiments, the catalyst is Pd(TFA)₂. In some embodiments, the catalyst is Pd₂(dba)₃. In some embodiments, the catalyst is Pd₂(dba)₃·CHCl₃. In some embodiments, the catalyst is Pd(PPh₃)₄. In some embodiments, the catalyst is Pd(OAc)₂. In some embodiments, the catalyst is Pd(PCy₃)₂Cl₂. In some embodiments, the catalyst is Pd(PPh₃)₂Cl₂. In some embodiments, the catalyst is Pd[P(o-tol)₃]₂Cl₂. In some embodiments, the catalyst is Pd(amphos)Cl₂. In some embodiments, the catalyst is Pd(dppf)Cl₂. In some embodiments, the catalyst is Pd(dppf)Cl₂·CH₂Cl₂. In some embodiments, the catalyst is Pd(dtbpf)Cl₂. In some embodiments, the catalyst is Pd(MeCN)₄ (BF₄)₂. In some embodiments, the catalyst is PdCl₂. In some embodiments, the catalyst is XPhos-Pd-G3. In some embodiments, the catalyst is Pd-PEPPSI™-IPr. In some embodiments, the catalyst is Pd-PEPPSI™-SIPr. In some embodiments, the catalyst is Pd-PEPPSI™-IPent.

In some embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium hydroxide. In some embodiments, the base is cesium carbonate. In some embodiments, the base is potassium carbonate. In some embodiments, the base is sodium bicarbonate. In some embodiments, the base is sodium bicarbonate. In some embodiments, the base is piperidine. In some embodiments, the base is, 8-diazabicyclo[5.4.0]undec-7-ene. In some embodiments, the base is N,N-diisopropylethylamine. In some embodiments, the base is triethylamine.

In some embodiments, 3-halo-indole is reacted with N-vinyl succinimide under metal catalysis to provide the corresponding desired Heck product. Representative metal catalysts could include those of palladium, nickel, ruthenium, iridium, etc. Preferred metal catalysts include Pd(OAc)₂. The intermediate 3-vinylindole is hydrogenated and the succinimide group is removed with base, e.g. KOH. The primary amine is methylated under Eschweiler-Clarke conditions (HCHO, HCO₂H) or similar reductive amination methods (HCHO, NaCNBH₃; HCHO, NaBH₄), or by methylation (MeI, NaHCO₃). The obtained psilocin (I-4) is derivatized to psilocybin (1-5) or O-acetylpsilocin (1-6), as illustrated in Schemes 1 and 3 above.

Reference is made to Yi et al., Chemical Research in Chinese Universities, 1996, 12, 136-141.

The Heck-type methodology outlined in Scheme 3 has the advantage of avoiding highly reactive and toxic reagents such as oxalyl chloride, which are a feature of other known synthetic routes for psilocin. Additionally, the route avoids highly reactive reducing agents, such as lithium aluminum hydride (LAH).

### 3. Conjugate Addition of an Indole to Nitroethylene or a Synthon for Nitroethylene

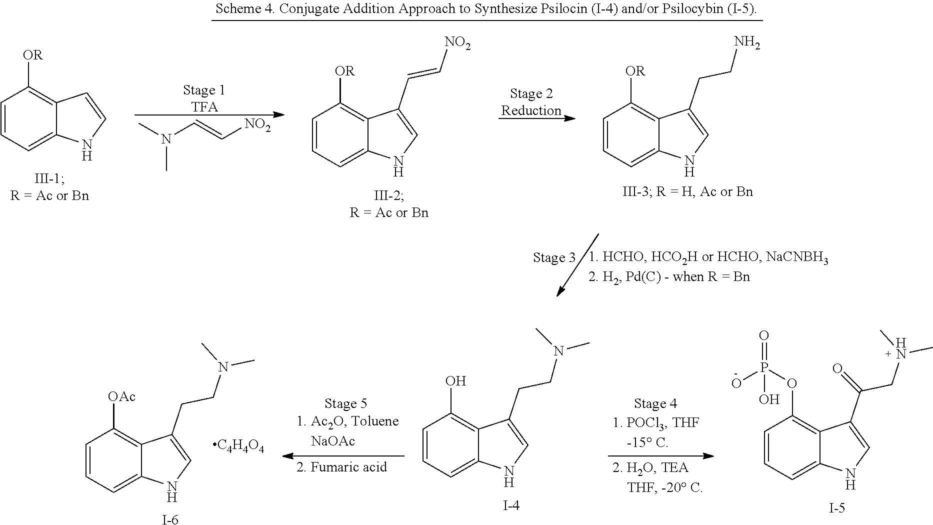

In some embodiments, psilocin and psilocybin can be synthesized using the synthetic route illustrated in Scheme 4 via making a nitroethylene intermediate or synthon.

In another aspect, provided herein are process for preparing Compound 1 (psilocin):

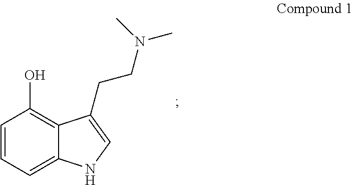

comprising (a) contacting Compound G:

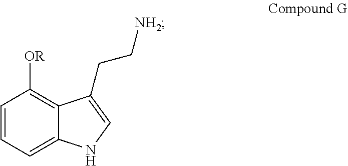

with a formaldehyde in the presence of a reducing agent, wherein R is hydrogen, acetyl, or benzyl.

In some embodiments, the reducing agent is a boron-containing reducing agent. In some embodiments, the reducing agent is NaCNBH₃ or NaBH₄. In some embodiments, the reducing agent is formic acid.

In some embodiments, when R is benzyl, the processed further comprise (a′) contacting the product from step (a) with hydrogen gas in the presence of a catalyst.

In some embodiments, the catalyst is selected from Pd/C, Pd(OH)₂, Pd(OH)₂/C, Pd/Al₂O₃, Pd(OAc)₂/Et₃SiH, (PPh₃)₃RhCl, and PtO₂. In some embodiments, the catalyst is Pd/C. In some embodiments, the catalyst is Pd(OH)₂. In some embodiments, the catalyst is Pd(OH)₂/C. In some embodiments, the catalyst is Pd/Al₂O₃. In some embodiments, the catalyst is Pd(OAc)₂/Et₃SiH. In some embodiments, the catalyst is (PPh₃)₃RhCl. In some embodiments, the catalyst is PtO₂.

In some embodiments, the processed further comprise (b) contacting Compound F:

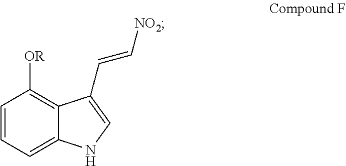

with a reducing agent in the presence of a solvent, wherein R is hydrogen, acetyl, or benzyl.

In some embodiments, the reducing agent is selected from metal borohydride, lithium aluminum hydride, diisobutyl aluminum hydride, and sodium borohydride-iodine or boranes. In some embodiments, the reducing agent is lithium aluminum hydride. In some embodiments, the reducing agent is diisobutyl aluminum hydride. In some embodiments, the reducing agent is sodium borohydride-iodine or boranes.

In some embodiments, the solvent is tetrahydrofuran, 2-Me-tetrahydrofuran, diethyl ether, dimethylformamide, dimethylsulfoxide, acetone, acetonitrile, 1,4-dioxane, or methyl tert-butyl ether, or combination thereof. In some embodiments, the solvent is tetrahydrofuran. In some embodiments, the solvent is, 2-Me-tetrahydrofuran. In some embodiments, the solvent is diethyl ether. In some embodiments, the solvent is dimethylformamide. In some embodiments, the solvent is dimethylsulfoxide. In some embodiments, the solvent is acetone. In some embodiments, the solvent is 1,4-dioxane. In some embodiments, the solvent is methyl tert-butyl ether.

In some embodiments, the processed further comprise (c) contacting Compound E:

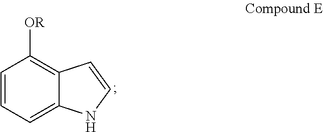

with (E)-N,N-dimethyl-2-nitroethen-1-amine in the presence of an acid to produce Compound F, wherein R is hydrogen, acetyl, or benzyl.

In some embodiments, the acid is trifluoroacetic acid.

In some embodiments, the processed further comprise (c) contacting Compound E:

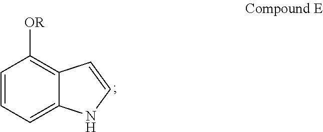

with POCl₃ in the presence of DMF to produce Compound F.

In some embodiments, the processed further comprise (c) contacting Compound E:

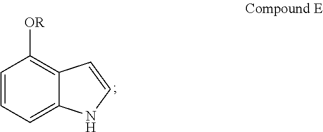

with Vilsmeir reagent (Me₂N⁺=CHCl Cl⁻) to produce Compound F.

In some embodiments, the preparation of intermediate III-2 involves formylation of a substituted indole derivative and a subsequent Henry reaction using NH₄OAc and MeNO₂ to provide the nitro-olefin III-2 (reference is made to Wiens et al., Tetrahedron, 2021, 81, 132055). Methods for the formylation of an appropriately substituted III-1 include use of a Vilsmeir-Haack reaction (POCl₃, DMF), reaction with the Vilsmeir reagent (Me₂N⁺=CHCl Cl⁻), reaction of HCHO, NH₃, FeCl₃ in DMF under air, 4-methoxy-N,N-dimethylaniline, DDQ, pivalic acid, DMF, 2,4,6-trichloro-1,3,5-triazine, hexamethylenetetramine (HMTA) and silica-supported ceric ammonium nitrate (CAN-SiO₂), NH₄OAc and DMSO, and other suitable reagent.

Reference is made to Repke et al., Journal of Heterocyclic Chemistry, 1981, 175-178 and Repke et al., Journal of Heterocyclic Chemistry, 1982, 845-848, together with references cited in both papers.

The synthetic approach outlined in Scheme 4 has the advantage of avoiding toxic reagents such as oxalyl chloride, which is a feature of other known routes for the synthesis of psilocin.

### Exemplary Reaction to Produce (E)-3-(2-nitrovinyl)-1H-indol-4-yl acetate (III-2, R=Ac)

1H-indol-4-yl acetate (III-1, R=Ac, 151 mg, 861 μmol) was reacted with (E)-N,N-dimethyl-2-nitroethen-1-amine (100 mg, 861 μmol) in TFA (2 mL). The reaction mixture was quenched with NaHCO₃, extracted with MTBE, and produced 107 mg (50.5% yield) of the nitroalkene (III-2; R=Ac) following silica gel chromatography. This was reproduced on 5.00 g scale, yielding 4.17 g of intermediate III-2 (R=Ac).

### Exemplary Reaction to Produce 3-(2-aminoethyl)-1H-indol-4-ol (III-3, R=H)

To a solution of (E)-3-(2-nitrovinyl)-1H-indol-4-yl acetate (165 mg, 670 μmol) in THF (5 mL) was added LiAlH₄ in THF (127 mg, 3.35 mL, 1.0 M, 3.35 mmol) and the reaction was stirred at rt for 1 h. The mixture was placed in an oil bath at 60° C. and stirred at 60° C. overnight. The suspension was cooled to 0° C., then Na₂SO₄ (3 g) in H₂O (5 mL) was added dropwise until no further effervescence was observed. The suspension turned dark green in color and gave a nicely filterable suspension. The suspension was filtered over a pad of celite and the filter cake was washed with 2Me-THF (2×20 mL). The combined filtrate was concentrated under reduced pressure to give 3-(2-aminoethyl)-1H-indol-4-ol (III-3, R=H, 72.1 mg, 61%) as a dark oil.

### Exemplary Reaction to Produce (E)-4-(benzyloxy)-3-(2-nitrovinyl)-1H-indole (III-2, R=Bn)

To 4-(benzyloxy)-1H-indole (III-1, R=Bn, 10.0 g, 44.8 mmol) in TFA (148 g, 100 mL, 1.30 mol) at 0° C. was added (E)-N,N-dimethyl-2-nitroethen-1-amine (5.20 g, 44.8 mmol). After complete addition, the mixture was allowed to warm to rt and stirred at rt for 24 h. The mixture was cooled to 0° C. and NaHCO₃ (132 g, 1.57 mol) in H2O (1 L) was added dropwise with MTBE (300 mL), making sure the internal temperature did not exceed 30° C. The organic layer was separated, and the aqueous layer extracted with MTBE (2×300 mL). The combined organic layers were concentrated under reduced pressure and adsorbed onto silica gel, then purified by column chromatography on silica gel using EtOAc/Hexanes (0:1 to 3:7) as eluent to give (E)-4-(benzyloxy)-3-(2-nitrovinyl)-1H-indole (III-2, R=Bn, 8.68 g, 66%).

### 4. Mannich Reaction of a 3-methylindole

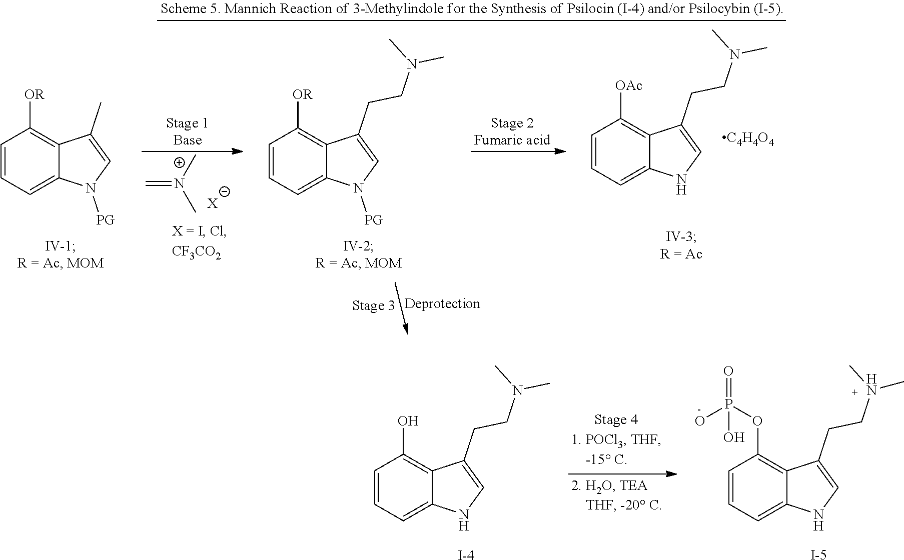

In some embodiments, psilocin and psilocybin can be synthesized using the synthetic route illustrated in Scheme 5 using a Mannich reaction.

In another aspect, provided herein are process for preparing Compound 1 (psilocin):

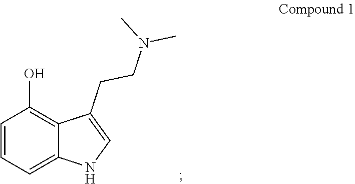

comprising (a) contacting Compound H:

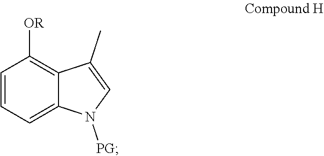

with Mannich salt Me₂N⁺=CH₂ X⁻ in the presence of a base, wherein X is I, Cl or CF₃CO₂, R is acetyl or methoxymethyl (MOM), and PG is a protecting group;
(b) removing the protecting group in Compound J:

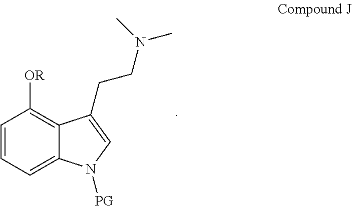

In some embodiments, the protecting group is Boc, tosyl, or methoxymethyl (MOM). In some embodiments, the protecting group is Boc. In some embodiments, the protecting group is tosyl. In some embodiments, the protecting group is methoxymethyl (MOM).

In some embodiments, the base is nBuLi, BuLi, MeMgBr, or RAMgCl·LiCl, wherein R^{A} is alkyl or N-containing hetercyclyl. In some embodiments, the base is nBuLi. In some embodiments, the base is fBuLi. In some embodiments, the base is MeMgBr. In some embodiments, the base is R^{A}MgCl·LiCl, wherein R^{A} is alkyl or N-containing hetercyclyl. In some embodiments, the base is iPrMgCl·LiCl, 2,2,6,6-tetramethylpiperidine, or sBuMgCl·LiCl.

In some embodiments, the base is a Grignard or a Turbo Grignard reagent.

In some embodiments, a preformed Mannich salt can be reacted with the anion of 3-methylindole to provide a tryptamine. In some embodiments of the Mannich salt Me₂N⁺=CH₂ X⁻, X is I, Cl or CF₃CO₂. In some embodiments, the salt can also be prepared by reaction of Me₂NCH₂NMe₂ and TMSI. In some embodiments, R is methoxymethyl (MOM) for the stabilization of the anion of 3-methylindole. Suitable indole nitrogen protecting groups (PG) include Boc, tosyl (Ts), methoxymethyl (MOM), amongst others. The base can be nBuLi, BuLi, MeMgBr, “Turbo-Grignard” reagents, RMgCl·LiCl, e.g. iPrMgCl·LiCl, TMPMgCl·LiCl (TMP=2,2,6,6-tetramethylpiperidine), sBuMgCl·LiCl, etc.

Reference is made to Holy, Synthetic Communications, 1976, 6, 539-542 and Bryson et al., The Journal of Organic Chemistry 1980, 45, 524-525 for the reaction of Mannich salts with an organometallic reagent.

The synthetic route as illustrated in Scheme 5 avoids toxic and highly reactive reagents such as oxalyl chloride, and highly reactive and pyrophoric reagents such as LAH.

### 5. Reaction of an Indole with an Aziridinium Species or Equivalent

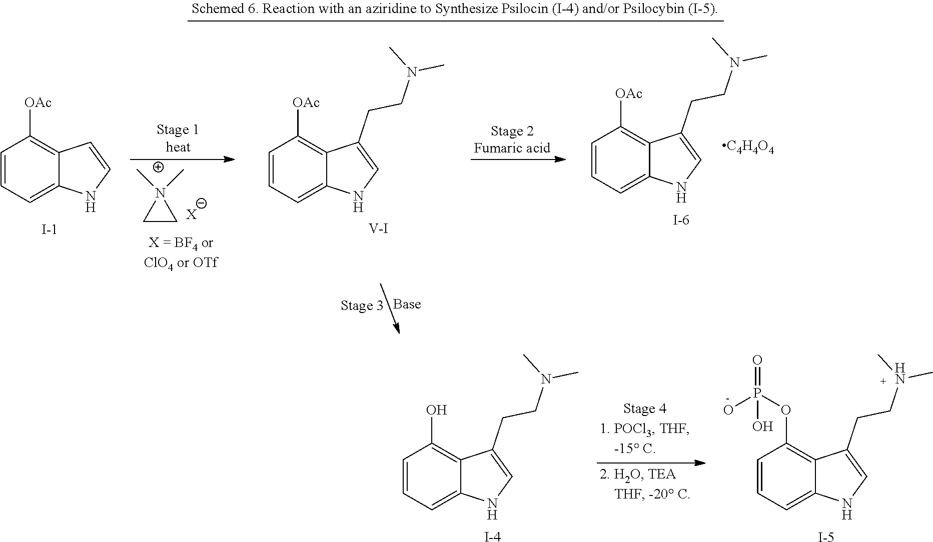

In some embodiments, psilocin and psilocybin can be synthesized using the synthetic routes illustrated in Scheme 6 or Scheme 7 using an aziridinium species.

In another aspect, provided herein are process for preparing Compound 1 (psilocin):

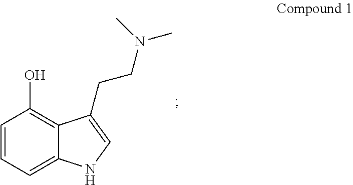

comprising (a) contacting Compound K:

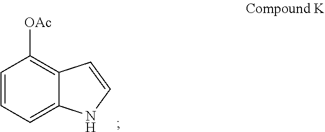 [SMILES: CC(=O)Oc1cccc2[nH]ccc12]

with a 1,1-dimethylaziridinium compound; and
(b) contacting Compound L:

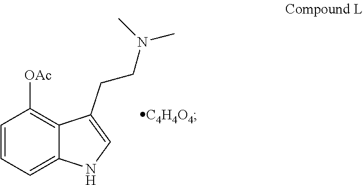

with a base in the presence of a solvent.

In some embodiments, the 1,1-dimethylaziridinium compound is 1,1-dimethylaziridinium BF₄⁻, ClO₄⁻, or OTf⁻. In some embodiments, the 1,1-dimethylaziridinium compound is 1,1-dimethylaziridinium BF₄. In some embodiments, the 1,1-dimethylaziridinium compound is 1,1-dimethylaziridinium ClO₄⁻. In some embodiments, the 1,1-dimethylaziridinium compound is 1,1-dimethylaziridinium OTf⁻.

In some embodiments, the 1,1-dimethylaziridinium compound is produced in situ by contacting Me₂NCH₂CH₂Cl·HCl and a second base under heating condition. In some embodiments, the second base is an inorganic base selected from Cs₂CO₃, K₂CO₃, Na₂CO₃, or NaHCO₃, or the second base is an organic base selected from piperidine, 1,8-diazabicyclo[5.4.0]undec-7-ene, N,N-diisopropylethylamine, and triethylamine.

In some embodiments, wherein the 1,1-dimethylaziridinium compound is produced in situ from Me₂NCH₂CH₂OMs under heating condition.

In some embodiments, step (a) occurs at a temperature between 20° C. and 150° C. In some embodiments, step (a) occurs at a temperature between 50° C. and 120° C. In some embodiments, step (a) occurs at a temperature between 70° C. and 100° C.

In some embodiments, the processes further comprise (b′) contacting with fumaric acid.

In some embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium hydroxide. In some embodiments, the base is cesium carbonate. In some embodiments, the base is potassium carbonate. In some embodiments, the base is sodium bicarbonate. In some embodiments, the base is sodium bicarbonate. In some embodiments, the base is piperidine. In some embodiments, the base is, 8-diazabicyclo[5.4.0]undec-7-ene. In some embodiments, the base is N,N-diisopropylethylamine. In some embodiments, the base is triethylamine.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the tryptamine intermediate V-1 is formed from a reaction of an indole with an aziridinium tetrafluoroborate. In some embodiments, the reaction in Stage 1 can be heated from room temperature (about 20° C.) to 150° C. In some embodiments, the most favorable temperature range is 50° C. to 120° C., with 70° C. to 100° C. being optimal. In some embodiments, 1,1-dimethylaziridinium species can also be formed in situ by employing Me₂NCH₂CH₂Cl·HCl and 1.0 equivalent of base under the reaction conditions. In some embodiments, the base can be an inorganic base such as Cs₂CO₃, K₂CO₃, Na₂CO₃, NaHCO₃, etc. or an organic base such as Et₃N, ⁱPr₂NEt, etc. Other reagents to form 1,1-dimethylaziridinium species in situ can include Me₂NCH₂CH₂OMs and heating. Reference is made to Pfeil et al., Angewante Chemie International Edition in English, 1967, 6, 178 for the general reaction of an indole with an aziridinium tetrafluoroborate to provide a tryptamine product. Reference is also made to Rinehart et al., J. Am. Chem. Soc., 1987, 109, 3378-3387 for reaction of an indole with aziridinium tetrafluoroborate to provide a tryptamine product. Reference is also made to Di Vona et al., Journal of The Chemical Society, Perkin Transactions II, 1985, 1943-1946 for the preparation of 1,1-dimethylaziridinium species.

Alternatively, as illustrated in Schemed 7 below, an appropriately substituted indole can be contacted with aziridinium tetrafluoroborate to provide an appropriately substituted intermediate III-3 (reference is made to Pfeil et al., Angewante Chemie International Edition in English, 1967, 6, 178 and Rinehart et al., J. Am. Chem. Soc., 1987, 109, 3378-3387). The reaction in Stage 1 can be heated from room temperature (20° C.) to 150° C. In some embodiments, the most favorable temperature range is 50° C. to 120° C., with 70° C. to 100° C. being optimal. In some embodiments, intermediate III-3 is methylated under Eschweiler-Clarke conditions (HCHO, HCO₂H) or similar reductive amination methods (HCHO, NaCNBH₃; HCHO, NaBH₄), or by methylation (MeI, NaHCO₃).

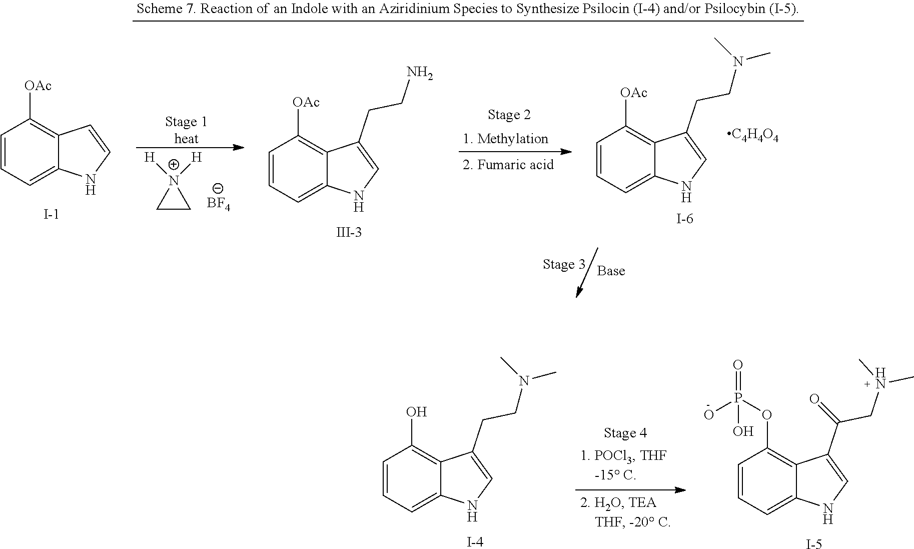

### 6. Reaction of an Indole with a Cyclic Sulfamidate

As showed in Scheme 8 below, psilocin and psilocybin can also be synthesized using a cyclic sulfamidate.

In another aspect, provided herein are process for preparing Compound 1 (psilocin):

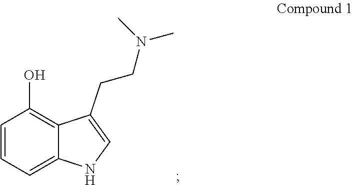

comprising (a) contacting Compound K:

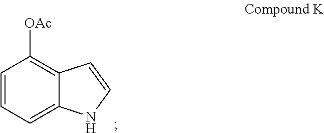 [SMILES: CC(=O)Oc1cccc2[nH]ccc12]

with aziridinium tetrafluoroborate

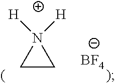

and
(b) contacting Compound M:

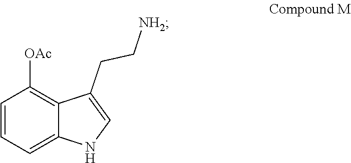 [SMILES: CC(=O)Oc1cccc2[nH]cc(CCN)c12]

with a formaldehyde in the presence of a reducing agent, or with iodomethane in the presence of a first base; and
(c) contacting Compound L:

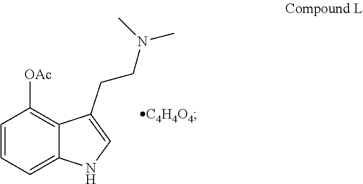

with a second base in the presence of a solvent.

In some embodiments, the processes further comprise (b′) contacting with fumaric acid.

In some embodiments, in step (b) the reducing agent is a boron-containing reducing agent. In some embodiments, the reducing agent is NaCNBH₃ or NaBH₄.

In some embodiments, the first base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the first base is sodium hydroxide. In some embodiments, the first base is potassium hydroxide. In some embodiments, the first base is cesium carbonate. In some embodiments, the first base is potassium carbonate. In some embodiments, the first base is sodium bicarbonate. In some embodiments, the first base is sodium bicarbonate. In some embodiments, the first base is piperidine. In some embodiments, the first base is, 8-diazabicyclo[5.4.0]undec-7-ene. In some embodiments, the first base is N,N-diisopropylethylamine. In some embodiments, the first base is triethylamine.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, an appropriately substituted indole can be reacted with a cyclic sulfamidate to provide a protected tryptamine, that can be deprotected to provide intermediate III-3. In some embodiments, favored metal bases and additives for the ring-opening reaction include MeMgBr and CuCl in DCM at −20° C. to 0° C.

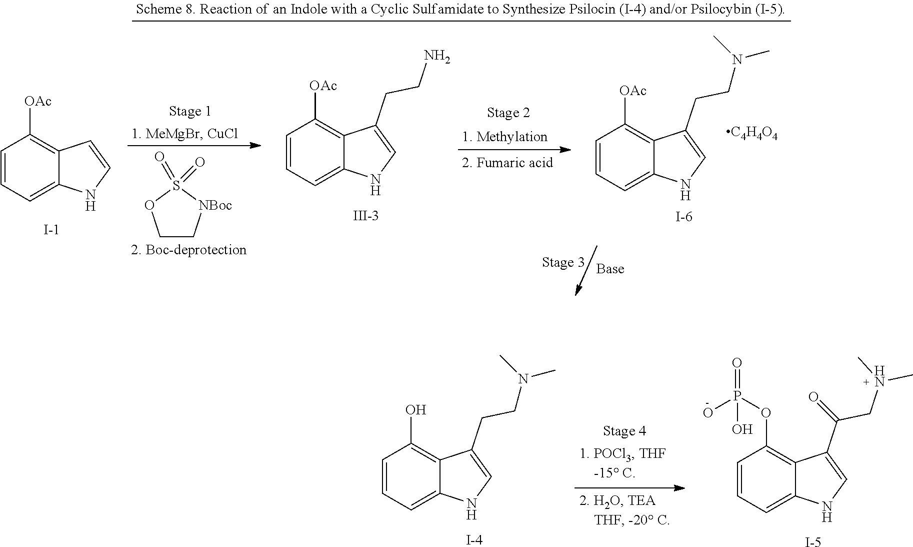

Reference is made to J. Wolfard et al., Org. Lett., 2018, 20, 5431 and references cited therein.

The synthetic routes illustrated in Schemes 6-8 have several advantages to current state-of-the-art for the synthesis of psilocin. For example, the chemistry outlined in Scheme 6 can potentially prepare psilocin in one step from 4-acetoxyindole. The aziridinium ring-opening reaction outlined in Scheme 6 is atom economical and may even exclude solvent. Thus, waste streams would be minimized for this synthesis. Additionally, the chemistry outlined in Schemes 7 and 8 are also shorter than established routes to psilocin, and relatives. The chemistry in Schemes 6-8 also avoids toxic and highly reactive reagents such as oxalyl chloride, and highly reactive and pyrophoric reagents such as LAH.

### 7. Reaction Using 2-(dimethylamino)acetaldehyde, protected 2-(dimethylamino)acetaldehyde or 2-dialkylaminoethanol

In some embodiments, psilocin and psilocybin can be synthesized using the synthetic routes illustrated in Scheme 9 (using 2-(dimethylamino)acetaldehyde) or Scheme 10 (using 2-dialkylaminoethanol).

In another aspect, provided herein are process for preparing Compound 1 (psilocin):

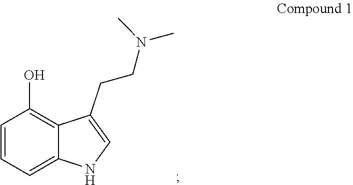

comprising (a) contacting Compound K:

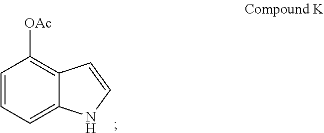 [SMILES: CC(=O)Oc1cccc2[nH]ccc12]

with a cyclic sulfamidate in the presence of a metal base; and
(b) contacting Compound M:

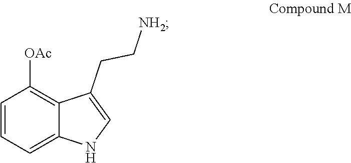 [SMILES: CC(=O)Oc1cccc2[nH]cc(CCN)c12]

with a formaldehyde in the presence of a reducing agent, or with iodomethane in the presence of a first base; and
(c) contacting Compound L:

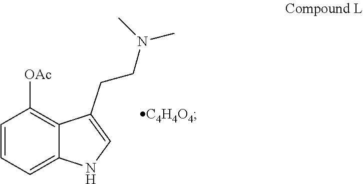

with a second base in the presence of a solvent.

In some embodiments, step (a) occurs at a temperature between 20° C. to 150° C. In some embodiments, step (a) occurs at a temperature between 50° C. to 120° C. In some embodiments, step (a) occurs at a temperature between 70° C. to 100° C.

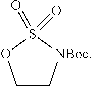 [SMILES: CC(C)(C)OC(=O)N1CCOS1(=O)=O]

In some embodiments, the cyclic sulfamidate is

In some embodiments, the metal base is MeMgBr.

In some embodiments, step (a) occurs in the presence of MeMgBr and CuCl in DCM at −20° C. to 0° C.

In some embodiments, the processes further comprise (a′) deprotecting Boc protecting group.

In some embodiments, in step (b) the reducing agent is a boron-containing reducing agent. In some embodiments, the reducing agent is NaCNBH₃ or NaBH₄.

In some embodiments, the first base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the first base is sodium hydroxide. In some embodiments, the first base is potassium hydroxide. In some embodiments, the first base is cesium carbonate. In some embodiments, the first base is potassium carbonate. In some embodiments, the first base is sodium bicarbonate. In some embodiments, the first base is sodium bicarbonate. In some embodiments, the first base is piperidine. In some embodiments, the first base is, 8-diazabicyclo[5.4.0]undec-7-ene. In some embodiments, the first base is N,N-diisopropylethylamine. In some embodiments, the first base is triethylamine.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the processes further comprise (b′) contacting with fumaric acid.

In some embodiments, the second base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the second base is sodium hydroxide. In some embodiments, the second base is potassium hydroxide. In some embodiments, the second base is cesium carbonate. In some embodiments, the second base is potassium carbonate. In some embodiments, the second base is sodium bicarbonate. In some embodiments, the second base is sodium bicarbonate. In some embodiments, the second base is piperidine. In some embodiments, the second base is, 8-diazabicyclo[5.4.0]undec-7-ene. In some embodiments, the second base is N,N-diisopropylethylamine. In some embodiments, the second base is triethylamine.

The reaction of 2-(dimethylamino)acetaldehyde, or a protected 2-(dimethylamino)acetaldehyde, such as 2-(dimethylamino)acetaldehyde sulfite (CAS No: 1413945-87-5), can be reacted with an appropriately substituted indole and the resulting alcohol (or enamine) reduced thereafter, for example, using NaBH₄. It is also possible to employ other protected versions of 2-(dimethylamino)acetaldehyde, such as (dimethylamino)acetaldehyde dimethyl acetal (CAS No: 38711-20-5). The reaction of the appropriately substituted indole with 2-(dimethylamino)acetaldehyde, or a protected version of 2-(dimethylamino)acetaldehyde may require the assistance of a Lewis acid catalyst, e.g. AlCl₃, BF₃₀Et₂, ZnCl₂, Zn(OTf)₂, Sc(OTf)₃, CuCl₂, etc. or alternatively, use of a base such as MeMgBr combined with CuCl.

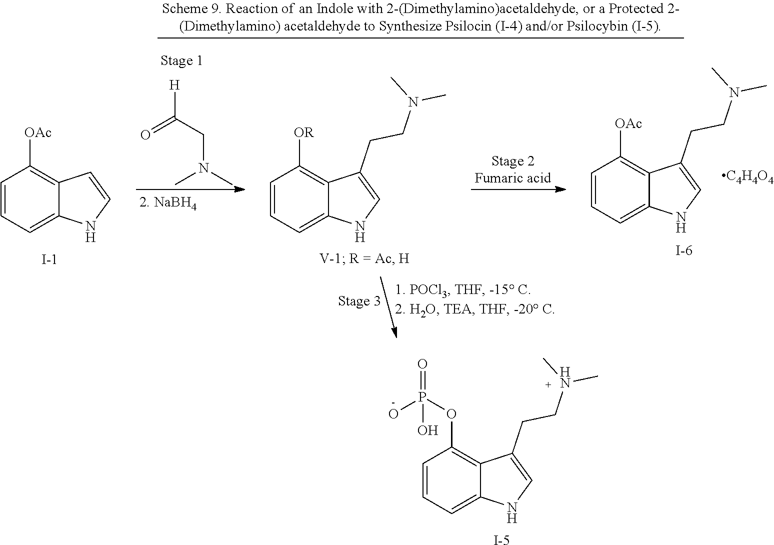

In an alternative process to prepare psilocin or psilocybin, 2-(dimethylamino) ethanol is used as a starting material in a “borrowing hydrogen” reaction, as shown in Scheme 10 below. Reference is made to Bartolucci et al., Tetrahedron, 2016, 72, 2233-2238 and Hall et al., Angew. Chem. Int. Ed., 2021, 60, 6981-6985. In certain embodiments, the catalyst for Stage 1 can be [Cp*IrCl₂]₂, Fe(II)Pc, Cu(OAc)₂/dppm, etc. In certain embodiments, the base can be Cs₂CO₃, NaO^{t}Bu, KO^{t}Bu, K₂CO₃, Na₂CO₃, NaHCO₃, etc. In certain embodiments, the reaction temperature can be 50° C. to 170° C., with a preferred range of 85° C. to 150° C.

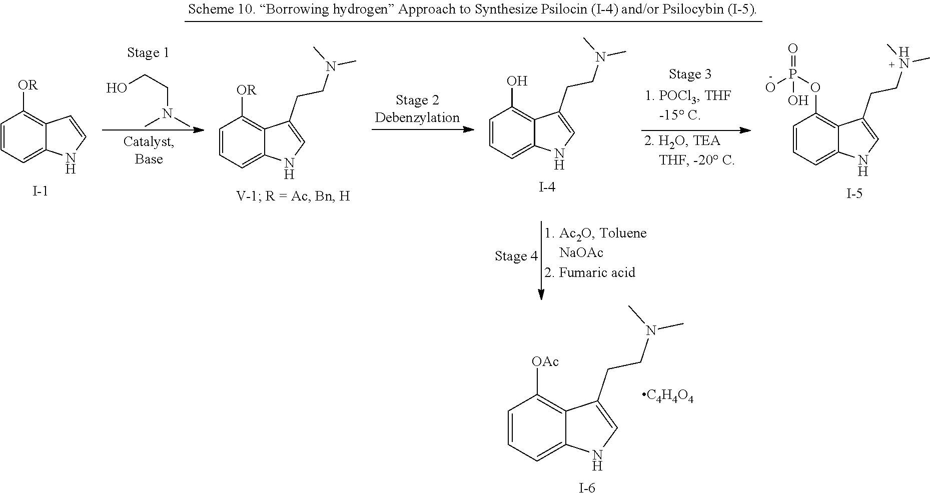

In another aspect, provided herein are process for preparing Compound 2 (psilocybin):

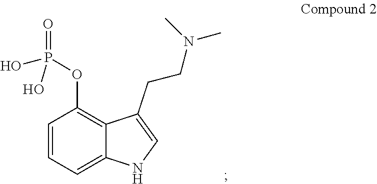 [SMILES: CN(C)CCc1c[nH]c2cccc(OP(=O)(O)O)c12]

comprising (a) contacting Compound K:

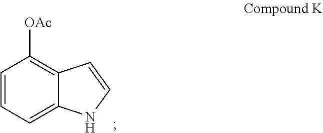 [SMILES: CC(=O)Oc1cccc2[nH]ccc12]

with 2-(dimethylamino)acetaldehyde or its derivative in the presence of a catalyst; and
(b) contacting Compound M:

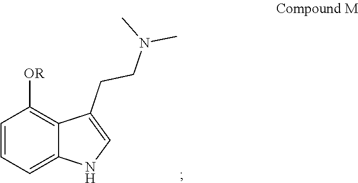

with POCl₃ in a solvent; wherein R is hydrogen or acetyl.

In some embodiments, the processes further comprise (c) contacting with water in the presence of a base in a solvent.

In some embodiments, the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium hydroxide. In some embodiments, the base is cesium carbonate. In some embodiments, the base is potassium carbonate. In some embodiments, the base is sodium bicarbonate. In some embodiments, the base is sodium bicarbonate. In some embodiments, the base is piperidine. In some embodiments, the base is, 8-diazabicyclo[5.4.0]undec-7-ene. In some embodiments, the base is N,N-diisopropylethylamine. In some embodiments, the base is triethylamine.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the catalyst is a Lewis acid catalyst. In some embodiments, the Lewis acid catalyst is AlCl₃, BF₃OEt₂, NaBH₄, ZnCl₂, Zn(OTf)₂, Sc(OTf)₃, or CuCl₂. In some embodiments, the Lewis acid catalyst is AlCl₃. In some embodiments, the Lewis acid catalyst is BF₃OEt₂. In some embodiments, the Lewis acid catalyst is NaBH₄. In some embodiments, the Lewis acid catalyst is ZnCl₂. In some embodiments, the Lewis acid catalyst is Zn(OTf)₂. In some embodiments, the Lewis acid catalyst is Sc(OTf)₃. In some embodiments, the Lewis acid catalyst is.

In some embodiments, the catalyst is a base. In some embodiments, the catalyst is MeMgBr combined with CuCl.

In some embodiments, the 2-(dimethylamino)acetaldehyde derivative is (dimethylamino)acetaldehyde dimethyl acetal.

In some embodiments, step (a) and step (b) occur in one-pot synthesis.

In another aspect, provided herein are process for preparing Compound 1 (psilocin):

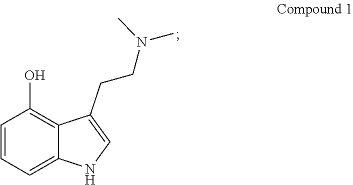

comprising (a) contacting Compound E:

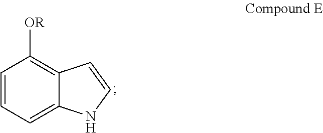

with 2-(dimethylamino) ethanol in the presence of a first catalyst and a first base, wherein R is hydrogen, acetyl, or benzyl; and
(b) contacting Compound N:

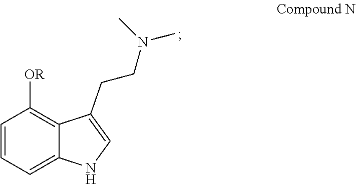

with hydrogen gas in the presence of a second catalyst, or with a second base.

The synthetic routes outlined in Schemes 9 and 10 have several advantages to current state-of-the-art for the synthesis of psilocin. For example, the chemistry outlined in Scheme 9 can potentially prepare psilocin in two steps from an appropriately substituted indole starting material. The two steps may also be able to be undertaken in a ‘one pot’ fashion. Additionally, the chemistry outlined in Scheme 10 is much shorter than established routes to psilocin. For instance, the chemistry in Scheme 10 can give psilocin in one step. The chemistry in Schemes 9 and 10 also avoids toxic and highly reactive reagents such as oxalyl chloride, and highly reactive and pyrophoric reagents such as LAH. Due to the shorter synthesis of psilocin and, or, psilocybin than established routes, the chemistry outlined in Schemes 9 and 10 may generate lower waste streams than established routes.

In some embodiments, the first catalyst is [Cp*IrCl₂]₂, Fe(II)Pc, or Cu(OAc)₂/dppm. In some embodiments, the first catalyst is [Cp*IrCl₂]₂. In some embodiments, the first catalyst is Fe(II)Pc. In some embodiments, the first catalyst is Cu(OAc)₂/dppm.

In some embodiments, the first base is Cs₂CO₃, NaO^{t}Bu, KO^{t}Bu, K₂CO₃, Na₂CO₃, or NaHCO₃. In some embodiments, the first base is NaHCO₃.

In some embodiments, step (a) occurs at a temperature of between 50° C. and 170° C. In some embodiments, wherein step (a) occurs at a temperature of between 85° C. and 150° C.

In some embodiments, the second base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the second base is sodium hydroxide. In some embodiments, the second base is potassium hydroxide. In some embodiments, the second base is cesium carbonate. In some embodiments, the second base is potassium carbonate. In some embodiments, the second base is sodium bicarbonate. In some embodiments, the second base is sodium bicarbonate. In some embodiments, the second base is piperidine. In some embodiments, the second base is, 8-diazabicyclo[5.4.0]undec-7-ene. In some embodiments, the second base is N,N-diisopropylethylamine. In some embodiments, the second base is triethylamine.

In some embodiments, the second catalyst is selected from Pd/C, Pd(OH)₂, Pd(OH)₂/C, Pd/Al₂O₃, Pd(OAc)₂/Et₃SiH, (PPh₃)₃RhCl, and PtO₂. In some embodiments, the second catalyst is Pd/C. In some embodiments, the second catalyst is Pd(OH)₂. In some embodiments, the second catalyst is Pd(OH)₂/C. In some embodiments, the second catalyst is Pd/Al₂O₃. In some embodiments, the second catalyst is Pd(OAc)₂/Et₃SiH. In some embodiments, the second catalyst is (PPh₃)₃RhCl. In some embodiments, the second catalyst is PtO₂.

### 8. Mannich Reaction Followed by Rearrangement

In some embodiments, psilocin and psilocybin can be synthesized using the synthetic routes illustrated in Scheme 11 using a Mannich reaction followed by an rearrangement.

In some embodiments, a Mannich reaction of an appropriately substituted indole with chloroacetaldehyde and Me₂NH gives the Mannich product VI-1, that can be rearranged to the dimethyltryptamine product I-4, as illustrated in Scheme 11 below. Presumably, the rearrangement was through an aziridinium intermediate that is reduced to the tryptamine with, for example, NaBH₄.

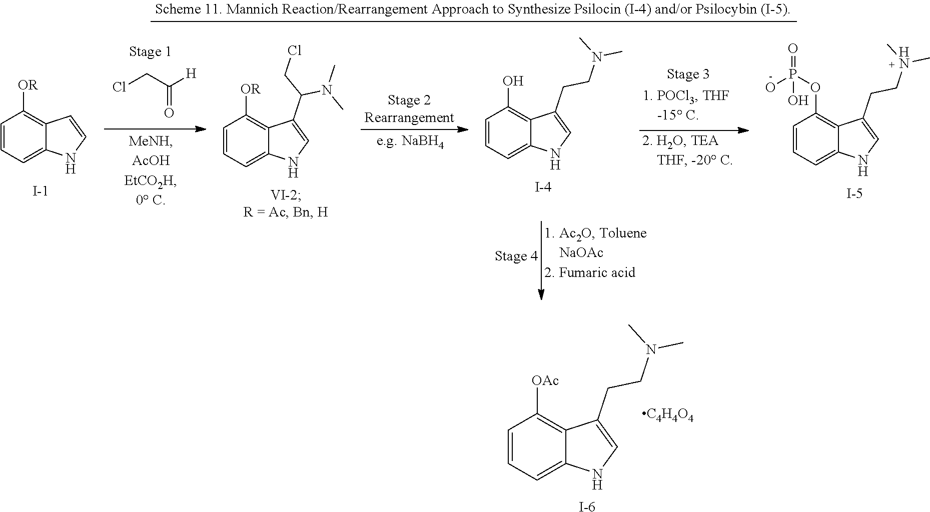

In another aspect, provided herein are process for preparing Compound 1 (psilocin):

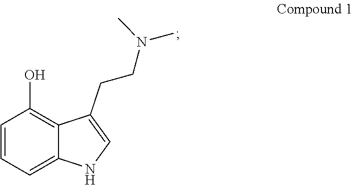

comprising (a) contacting Compound E:

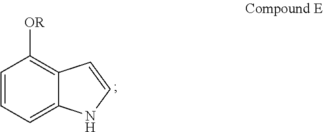

with chloroacetaldehyde and Me₂NH in the presence of an acid, wherein R is hydrogen, acetyl, or benzyl; and
(b) contacting Compound O:

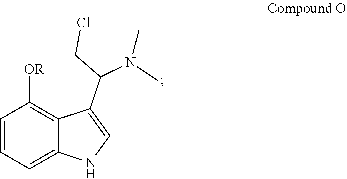

with a reducing agent.

In some embodiments, the acid is an organic acid. In some embodiments, the acid is acetic acid or propionic acid.

In some embodiments, step (a) occurs at a temperature of about 0° C.

In some embodiments, the reducing agent is a boron-containing reducing agent. In some embodiments, the reducing agent is NaCNBH₃ or NaBH₄.

In some embodiments, the first base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine. In some embodiments, the first base is sodium hydroxide. In some embodiments, the first base is potassium hydroxide. In some embodiments, the first base is cesium carbonate. In some embodiments, the first base is potassium carbonate. In some embodiments, the first base is sodium bicarbonate. In some embodiments, the first base is sodium bicarbonate. In some embodiments, the first base is piperidine. In some embodiments, the first base is, 8-diazabicyclo[5.4.0]undec-7-ene. In some embodiments, the first base is N,N-diisopropylethylamine. In some embodiments, the first base is triethylamine.

Reference is made to M. Julia et al., Bulletin de la Societe Chimique de France, 1973, 1424-1426.

The synthetic route as outlined in Scheme 11 also demonstrates a succinct approach to psilocin and/or psilocybin. Such a synthetic route has advantages over established methodology in that it is a shorter synthesis and may generate fewer waste streams. Additionally, the chemistry outlined in Scheme 11 avoids toxic and highly reactive reagents such as oxalyl chloride, and highly reactive and pyrophoric reagents such as LAH.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A process of preparing Compound 1: 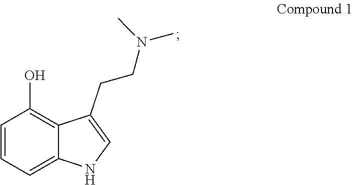
comprising (a-iii) contacting Compound D: 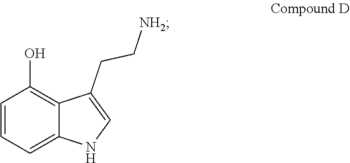 [SMILES: NCCc1c[nH]c2cccc(O)c12]
with a formaldehyde in the presence of a reducing agent, or with iodomethane in the presence of a base.

2. The process of claim 1, wherein the reducing agent is a boron-containing reducing agent.

3. The process of claim 2, wherein the reducing agent is NaCNBH₃ or NaBH₄.

4. The process of claim 1, wherein the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

5. The process of claim 4, wherein the base is sodium bicarbonate.

6. The process of any one of claims 1-5, further comprising (a-ii) contacting Compound C: 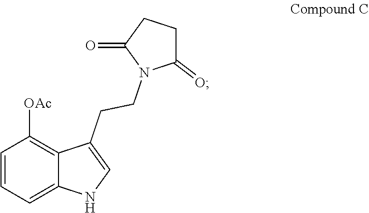 [SMILES: CC(=O)Oc1cccc2[nH]cc(CCN3C(=O)CCC3=O)c12]
with a base in the presence of a solvent to produce Compound D.

7. The process of claim 6, wherein the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

8. The process of claim 7, wherein the base is potassium hydroxide.

9. The process of any one of claims 1-8, further comprising (a-i) contacting Compound B: 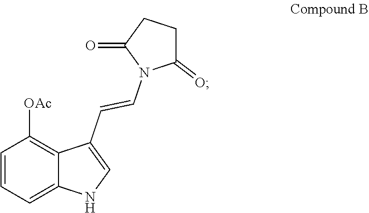 [SMILES: CC(=O)Oc1cccc2[nH]cc(/C=C/N3C(=O)CCC3=O)c12]
with a reducing agent in the presence of a catalyst and a solvent.

10. The process of claim 9, wherein the reducing agent is hydrogen gas.

11. The process of claim 9 or 10, wherein the catalyst is selected from Pd/C, Pd(OH)₂, Pd(OH)₂/C, Pd/Al₂O₃, Pd(OAc)₂/Et₃SiH, (PPh₃)₃RhCl, and PtO₂.

12. The process of any one of claims 1-11, further comprising (b) contacting Compound A: 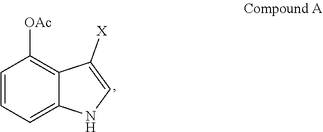
with N-vinyl succinimide in the presence of a catalyst, a base, and a solvent; wherein X is a halo.

13. The process of claim 12, wherein the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(MeCN)₄ (BF₄)₂, PdCl₂, XPhos-Pd-G3, Pd-PEPPSI™-IPr, Pd-PEPPSI™-SIPr, and Pd-PEPPSI™-IPent.

14. The process of claim 13, wherein the catalyst is Pd(OAc)₂.

15. The process of claim 13 or 14, wherein the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

16. The process of claim 13 or 14, wherein the base is triethylamine.

17. A process of preparing Compound 1: 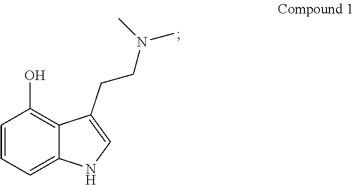
comprising (a) contacting Compound G: 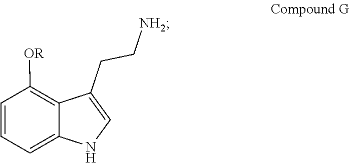
with a formaldehyde in the presence of a reducing agent, wherein R is hydrogen, acetyl, or benzyl.

18. The process of claim 17, wherein the reducing agent is a boron-containing reducing agent.

19. The process of claim 17 or 18, wherein the reducing agent is NaCNBH₃ or NaBH₄.

20. The process of claim 17, wherein the reducing agent is formic acid.

21. The process of claim 17, wherein when R is benzyl, further comprising (a′) contacting the product from step (a) with hydrogen gas in the presence of a catalyst.

22. The process of claim 21, wherein the catalyst is selected from Pd/C, Pd(OH)₂, Pd(OH)₂/C, Pd/Al₂O₃, Pd(OAc)₂/Et₃SiH, (PPh₃)₃RhCl, and PtO₂.

23. The process of claim 21, wherein the catalyst is selected from Pd/C,

24. The process of any one of claims 17-23, further comprising (b) contacting Compound F: 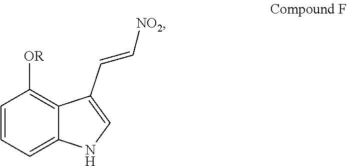
with a reducing agent in the presence of a solvent, wherein R is hydrogen, acetyl, or benzyl.

25. The process of claim 24, wherein the reducing agent is selected from metal borohydride, lithium aluminum hydride, diisobutyl aluminum hydride, and sodium borohydride-iodine or boranes.

26. The process of claim 25, wherein the reducing agent is lithium aluminum hydride.

27. The process of any one of claims 24-26, wherein the solvent is tetrahydrofuran, 2-Me-tetrahydrofuran, diethyl ether, dimethylformamide, dimethylsulfoxide, acetone, acetonitrile, 1,4-dioxane, or methyl tert-butyl ether, or combination thereof.

28. The process of claim 27, wherein the solvent is tetrahydrofuran.

29. The process of any one of claims 17-28, further comprising: (c) contacting Compound E: 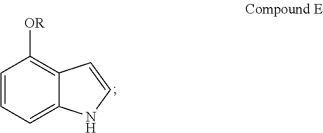
with (E)-N,N-dimethyl-2-nitroethen-1-amine in the presence of an acid to produce Compound F, wherein R is hydrogen, acetyl, or benzyl.

30. The process of claim 29, wherein the acid is trifluoroacetic acid.

31. The process of any one of claims 17-28, further comprising: (c) contacting Compound E: 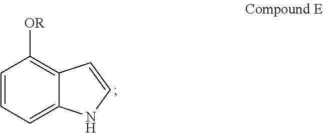
with POCl₃ in the presence of DMF to produce Compound F.

32. The process of any one of claims 17-28, further comprising: (c) contacting Compound E: 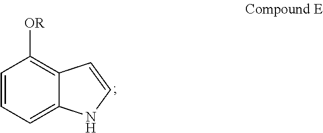
with Vilsmeir reagent (Me₂N⁺=CHCl Cl⁻) to produce Compound F.

33. A process of preparing Compound 1: 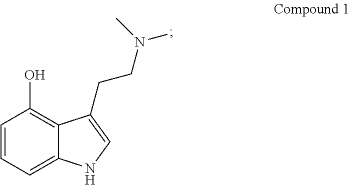
comprising (a) contacting Compound H: 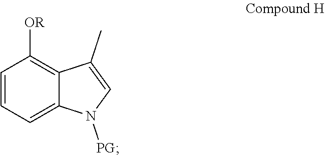
with Mannich salt Me₂N⁺=CH₂ X⁻ in the presence of a base, wherein X is I, Cl or CF₃CO₂, R is acetyl or methoxymethyl (MOM), and PG is a protecting group;
(b) removing the protecting group in Compound J:

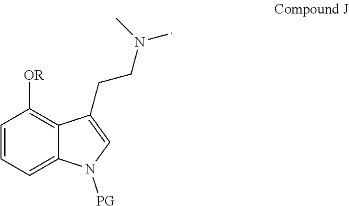

34. The process of claim 33, wherein the protecting group is Boc, tosyl, or methoxymethyl (MOM).

35. The process of claim 33 or 34, wherein the base is nBuLi, BuLi, MeMgBr, or R^{A}MgCl·LiCl, wherein R^{A} is alkyl or N-containing hetercyclyl.

36. The process of claim 35, wherein the base is iPrMgCl·LiCl, 2,2,6,6-tetramethylpiperidine, or sBuMgCl·LiCl.

37. The process of claim 33 or 34, wherein the base is a Grignard or a Turbo Grignard reagent.

38. A process of preparing Compound 1: 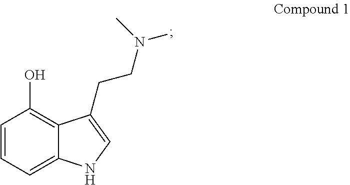
comprising (a) contacting Compound K: 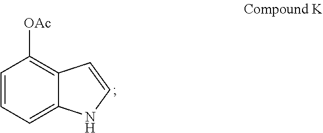 [SMILES: CC(=O)Oc1cccc2[nH]ccc12]
with a 1,1-dimethylaziridinium compound; and
(b) contacting Compound L: 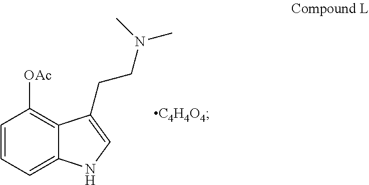
with a base in the presence of a solvent.

39. The process of claim 38, wherein the 1,1-dimethylaziridinium compound is 1,1-dimethylaziridinium BF₄⁻, ClO₄⁻, or OTf⁻.

40. The process of claim 38 or 39, wherein the 1,1-dimethylaziridinium compound is 1,1-dimethylaziridinium BF₄⁻.

41. The process of claim 40, wherein the 1,1-dimethylaziridinium compound is produced in situ by contacting Me₂NCH₂CH₂Cl·HCl and a second base under heating condition.

42. The process of claim 41, wherein the second base is an inorganic base selected from Cs₂CO₃, K₂CO₃, Na₂CO₃, or NaHCO₃, or the second base is an organic base selected from piperidine, 1,8-diazabicyclo[5.4.0]undec-7-ene, N,N-diisopropylethylamine, and triethylamine.

43. The process of claim 40, wherein the 1,1-dimethylaziridinium compound is produced in situ from Me₂NCH₂CH₂OMs under heating condition.

44. The process of any one of claims 38-42, wherein step (a) occurs at a temperature between 20° C. and 150° C.

45. The process of any one of claims 38-42, wherein step (a) occurs at a temperature between 50° C. and 120° C.

46. The process of any one of claims 38-42, wherein step (a) occurs at a temperature between 70° C. and 100° C.

47. The process of claim 38, further comprising (b′) contacting with fumaric acid.

48. The process of any one of claims 38-47, wherein the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

49. The process of claim 48, wherein the first base is potassium hydroxide.

50. A process of preparing Compound 1: 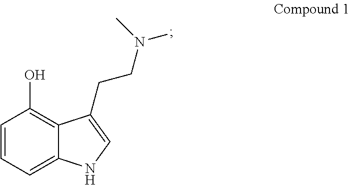
comprising (a) contacting Compound K: 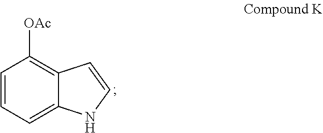 [SMILES: CC(=O)Oc1cccc2[nH]ccc12]
with aziridinium tetrafluoroborate 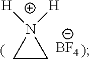
and
(b) contacting Compound M: 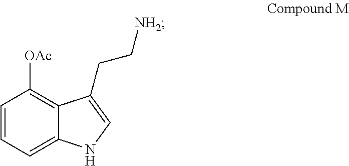 [SMILES: CC(=O)Oc1cccc2[nH]cc(CCN)c12]
with a formaldehyde in the presence of a reducing agent, or with iodomethane in the presence of a first base; and
(c) contacting Compound L: 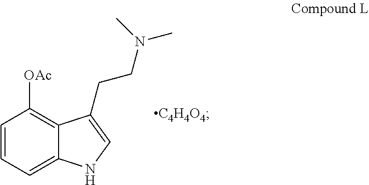
with a second base in the presence of a solvent.

51. The process of claim 50, further comprising (b′) contacting with fumaric acid.

52. The process of claim 50, wherein in step (b) the reducing agent is a boron-containing reducing agent.

53. The process of claim 51, wherein the reducing agent is NaCNBH₃ or NaBH₄.

54. The process of any one of claims 50-53, wherein the first base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

55. The process of claim 54, wherein the first base is sodium bicarbonate.

56. The process of any one of claims 50-55, wherein the second base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

57. The process of claim 56, wherein the second base is potassium hydroxide.

58. A process of preparing Compound 1: 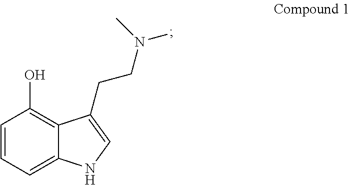
comprising (a) contacting Compound K: 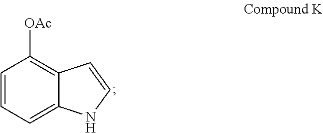 [SMILES: CC(=O)Oc1cccc2[nH]ccc12]
with a cyclic sulfamidate in the presence of a metal base; and
(b) contacting Compound M: 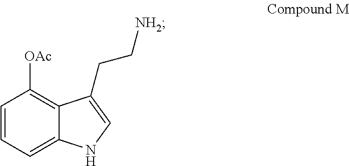 [SMILES: CC(=O)Oc1cccc2[nH]cc(CCN)c12]
with a formaldehyde in the presence of a reducing agent, or with iodomethane in the presence of a first base; and
(c) contacting Compound L: 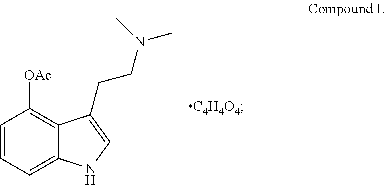 [SMILES: CC(=O)Oc1cccc2[nH]cc(CCN(C)C)c12]
with a second base in the presence of a solvent.

59. The process of claim 50, wherein step (a) occurs at a temperature between 20° C. to 150° C.

60. The process of claim 50, wherein step (a) occurs at a temperature between 50° C. to 120° C.

61. The process of claim 50, wherein step (a) occurs at a temperature between 70° C. to 100° C.

62. The process of claim 58, wherein the cyclic sulfamidate is

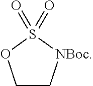 [SMILES: CC(C)(C)OC(=O)N1CCOS1(=O)=O]

63. The process of claim 58, wherein the metal base is MeMgBr.

64. The process of claim 58, wherein step (a) occurs in the presence of MeMgBr and CuCl in DCM at −20° C. to 0° C.

65. The process of claim 58, further comprising (a′) deprotecting Boc protecting group.

66. The process of any one of claims 50-65, wherein in step (b) the reducing agent is a boron-containing reducing agent.

67. The process of claim 66, wherein the reducing agent is NaCNBH₃ or NaBH₄.

68. The process of any one of claims 50-67, wherein the first base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

69. The process of claim 68, wherein the first base is sodium bicarbonate.

70. The process of claim 58, further comprising (b′) contacting with fumaric acid.

71. The process of any one of claims 50-70, wherein the second base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

72. The process of claim 71, wherein the second base is potassium hydroxide.

73. A process of preparing Compound 2: 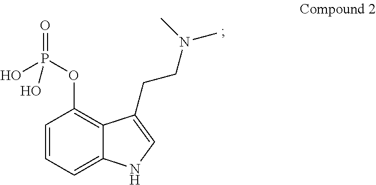 [SMILES: CN(C)CCc1c[nH]c2cccc(OP(=O)(O)O)c12]
comprising (a) contacting Compound K: 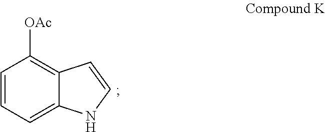 [SMILES: CC(=O)Oc1cccc2[nH]ccc12]
with 2-(dimethylamino)acetaldehyde or its derivative in the presence of a catalyst; and
(b) contacting Compound M: 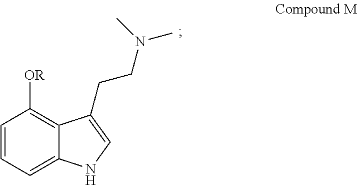
with POCl₃ in a solvent; wherein R is hydrogen or acetyl.

74. The process of claim 73, further comprising: (c) contacting with water in the presence of a base in a solvent.

75. The process of claim 74, wherein the base is triethylamine.

76. The process of any one of claims 73-75, wherein the catalyst is a Lewis acid catalyst.

77. The process of claim 76, wherein the Lewis acid catalyst is AlCl₃, BF₃OEt₂, NaBH₄, ZnCl₂, Zn(OTf)₂, Sc(OTf)₃, or CuCl₂.

78. The process of any one of claims 73-75, wherein the catalyst is a base.

79. The process of claim 78, wherein the catalyst is MeMgBr combined with CuCl.

80. The process of claim 73, wherein the 2-(dimethylamino)acetaldehyde derivative is (dimethylamino)acetaldehyde dimethyl acetal.

81. The process of claim 73, wherein step (a) and step (b) occur in one-pot synthesis.

82. A process of preparing Compound 1: 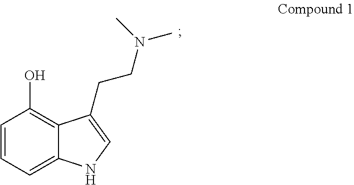
comprising (a) contacting Compound E: 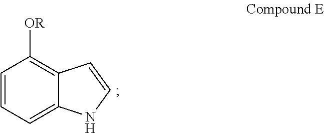
with 2-(dimethylamino) ethanol in the presence of a first catalyst and a first base, wherein R is hydrogen, acetyl, or benzyl; and
(b) contacting Compound N: 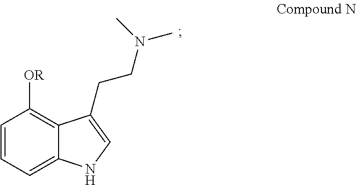
with hydrogen gas in the presence of a second catalyst, or with a second base.

83. The process of claim 82, wherein the first catalyst is [Cp*IrCl₂]₂, Fe(II)Pc, or Cu(OAc)₂/dppm.

84. The process of claim 82, wherein the first base is Cs₂CO₃, NaO^{t}Bu, KO^{t}Bu, K₂CO₃, Na₂CO₃, or NaHCO₃.

85. The process of any one of claims 82-84, wherein step (a) occurs at a temperature of between 50° C. and 170° C.

86. The process of any one of claims 82-84, wherein step (a) occurs at a temperature of between 85° C. and 150° C.

87. The process of any one of claims 82-86, wherein the first base is sodium bicarbonate.

88. The process of any one of claims 82-87, wherein the second base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

89. The process of claim 88, wherein the second base is potassium hydroxide.

90. The process of any one of claims 82-89, wherein the second catalyst is selected from Pd/C, Pd(OH)₂, Pd(OH)₂/C, Pd/Al₂O₃, Pd(OAc)₂/Et₃SiH, (PPh₃)₃RhCl, and PtO₂.

91. The process of claim 90, wherein the second catalyst is Pd/C.

92. A process of preparing Compound 1: 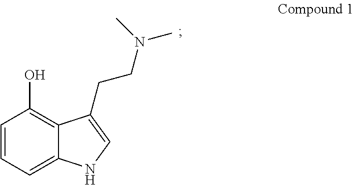
comprising (a) contacting Compound E: 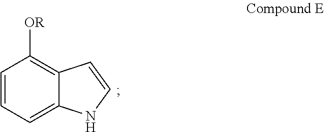
with chloroacetaldehyde and Me₂NH in the presence of an acid, wherein R is hydrogen, acetyl, or benzyl; and
(b) contacting Compound O: 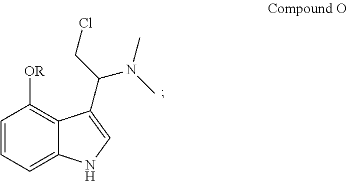
with a reducing agent.

93. The process of claim 92, wherein the acid is an organic acid.

94. The process of claim 92, wherein the acid is acetic acid or propionic acid.

95. The process of any one of claims 92-94, wherein step (a) occurs at a temperature of about 0° C.

96. The process of any one of claims 92-95, wherein the reducing agent is a boron-containing reducing agent.

97. The process of claim 96, wherein the reducing agent is NaCNBH₃ or NaBH₄.

98. A process of preparing Compound 2: 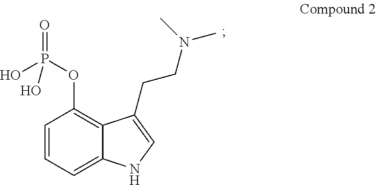 [SMILES: CN(C)CCc1c[nH]c2cccc(OP(=O)(O)O)c12]
comprising:
(a) preparing Compound 1 according to the process of any one of claims 1-72 and 82-97; and
(b) contacting Compound 1 with POCl₃ and a base in a solvent.

99. The process of claim 98, wherein the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

100. The process of claim 99, wherein the base is triethylamine.

101. The process of any one of claims 98-100, wherein the solvent is tetrahydrofuran, 2-Me-tetrahydrofuran, diethyl ether, dimethylformamide, dimethylsulfoxide, acetone, acetonitrile, 1,4-dioxane, or methyl tert-butyl ether, or combination thereof.

102. The process of claim 101, wherein the solvent is tetrahydrofuran

103. The process of any one of claims 98-102, wherein step (b) occurs at a temperature of about 0° C.

104. A process of preparing Compound 2: 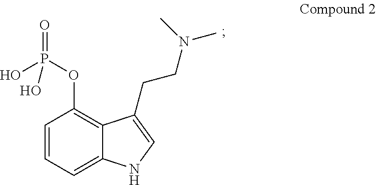 [SMILES: CN(C)CCc1c[nH]c2cccc(OP(=O)(O)O)c12]
comprising:
(a) preparing Compound 1 according to the process of any one of claims 1-72 and 82-97; and
(b) contacting Compound 1 with the Ψ-reagent, (2R,3aS,6R,7aS)-2-((4-bromophenyl)thio)-6-isopropyl-3a-methylhexahydrobenzo[d][1,3,2]oxathiaphosphole 2-oxide in the presence of base in a solvent.

105. The process of claim 93, wherein a stoichiometric amount of the ‘-reagent is contacted.

106. The process of claim 93, wherein between 1.0 and 10.0 equivalents of the Y’-reagent is contacted.

107. The process of any one of claims 93-106, wherein the base is selected from sodium hydroxide, potassium hydroxide, cesium carbonate, potassium carbonate, sodium carbonate, sodium bicarbonate, piperidine, 1,8-diazabicyclo[5.4.0]undec-7-ene, N,N-diisopropylethylamine, and triethylamine.

108. The process of claim 107, wherein the base is 1,8-diazabicyclo[5.4.0]undec-7-ene.

109. The process of claim 108, wherein between 1.0 and 10.0 equivalents of 1,8-diazabicyclo[5.4.0]undec-7-ene is present.

110. The process of any one of claims 104-109, wherein the solvent is tetrahydrofuran, 2-Me-tetrahydrofuran, dimethylformamide, dimethylsulfoxide, acetonitrile, dichloromethane, dichloroethane, chloroform, or toluene.

111. The process of claim 110, wherein the solvent is dimethylformamide.

112. The process of any one of claims 104-111, wherein step (b) occurs at a temperature of between room temperature and 150° C.

113. The process of any one of claims 104-112, further comprising (c) contacting water in the presence of acetonitrile.

114. A process of preparing Compound 2: 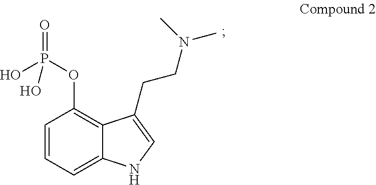 [SMILES: CN(C)CCc1c[nH]c2cccc(OP(=O)(O)O)c12]
comprising:
(a) preparing Compound 1 according to any one of claims 1-72 and 82-97; and
(b) contacting Compound 1 with phosphoric acid, Bu₃N, in the presence of a base in a solvent.

115. The process of claim 114, wherein between 1.0 and 10.0 equivalents of phosphoric acid is contacted.

116. The process of claim 114 or 115, wherein the base is a nucleophilic base.

117. The process of claim 116, wherein the nucleophilic base is N-butylimidazole or 4-(N,N-dimethylamino)pyridine.

118. The process of any one of claims 114-117, wherein a catalytic amount of the nucleophilic base is present.

119. The process of any one of claims 114-117, wherein up to a stoichiometric amount of the nucleophilic base is present.

120. The process of any one of claims 114-117, wherein between 10 mol % to 100 mol % of the nucleophilic base is present.

121. The process of any one of claims 114-120, wherein a stoichiometric amount of Bu₃N is present.

122. The process of any one of claims 114-120, wherein between 1.0 and 10.0 equivalents of Bu₃N is present.

123. The process of any one of claims 114-122, wherein the solvent is DMF, 1:1 (v/v) mixture of DMF and nitroethane, or o-xylene.

124. The process of any one of claims 114-123, wherein step (b) occurs at a temperature of between room temperature and 150° C.

125. The process of any one of claims 114-123, wherein step (b) occurs at a preferred temperature of azeotropic reflux with removal of water.

126. The process of claim 125, the removal of water uses 3 Å molecular sieves.
